(19) United States
(12) Patent Application Publication
(10) Pub. No.: US 2023/0013775 A1
(43) Pub. Date: Jan. 19, 2023

(54) METHODS FOR SAMPLE TRANSFER FOR IN SITU ANALYSIS

(71) Applicant: 10x Genomics, Inc., Pleasanton, CA (US)

(72) Inventors: Hong CHEN, Dublin, CA (US); Evan R. DAUGHARTHY, Cambridge, MA (US)

(21) Appl. No.: 17/812,075

(22) Filed: Jul. 12, 2022

Related U.S. Application Data
(60) Provisional application No. 63/221,455, filed on Jul. 13, 2021.

(51) Int. Cl. C12Q 1/6841; G01N 21/64; G01N 33/543

(52) U.S. Cl. CPC C12Q 1/6841; G01N 21/6428; G01N 33/54353

(57) ABSTRACT

The present disclosure relates in some aspects to methods for preparing biological samples for in situ analysis of one or more analytes, wherein the biological sample has been previously affixed to a substrate, which is not compatible with in situ analysis, for example, due to the absence of positional markers and/or fiducial markers and/or a region suitable for in situ signal detection on the substrate.

### CROSS-REFERENCE TO RELATED APPLICATION

This application claims the priority benefit of U.S. Provisional Patent Application Ser. No. 63/221,455, filed Jul. 13, 2021, the contents of which are incorporated herein by reference in their entirety.

### REFERENCE TO AN ELECTRONIC SEQUENCE LISTING

The contents of the electronic sequence listing (202412008800SEQLIST.xml; Size: 3,632 bytes; and Date of Creation: Jul. 7, 2022) is herein incorporated by reference in its entirety.

### FIELD

The present disclosure relates in some aspects to methods, compositions, kits, and devices for sample processing and analysis, such as compositions (e.g., a matrix-forming material or polymerized matrix), devices (e.g., a universal adapter), and methods of using the compositions and/or devices for transferring a sample for in situ analysis of analytes in the sample.

### BACKGROUND

Transcription profiling of cells is essential for many purposes, such as understanding the molecular basis of cell identity and/or function and developing treatments for diseases. Microscopic imaging techniques, which can resolve multiple mRNAs in single cells, can provide valuable information such as transcript abundance and spatial information in situ. Methods are available for analyzing nucleic acids present in a biological sample, such as a cell or a tissue. For instance, advances in single molecule fluorescence in situ hybridization (smFISH) have enabled nanoscale-resolution imaging of RNA in cells and tissues.

Existing methods of in situ analysis often employ particular substrates to which biological samples are affixed, such as specialized glass slides coated with oligonucleotide arrays which may bind to RNA present in the biological samples and/or modified with positioning and/or fiducial markers, in order to serve as part of the in situ analysis and facilitate downstream interpretation. However, out of convenience and availability, many biological samples may be affixed to substrates lacking these modifications. In other cases, there may be interest in applying in situ analytical techniques to archival biological samples that may have been originally intended for other techniques and immobilized to substrates incompatible with in situ methods. In such instances, the biological samples may not be readily examined with in situ analysis methods. Improved methods for sample processing and in situ analysis are needed. The present disclosure addresses these and other needs.

### SUMMARY

The present disclosure relates in some aspects to methods for preparing biological samples for in situ analysis of one or more analytes, wherein the biological sample has been previously affixed to a substrate, which is not compatible with in situ analysis.

In some aspects, provided herein are methods for preparing biological samples embedded in three-dimensional polymerized matrices (e.g., hydrogels) for sample processing and/or in situ analysis of one or more analytes (e.g., nucleic acid and/or protein molecules) in the biological sample embedded in the polymer matrix. In some embodiments, disclosed herein are methods of processing biological samples affixed to substrates that are not compatible with and/or not specialized for in situ analysis such as in situ transcriptomic profiling. In some embodiments, disclosed herein are methods of transferring a biological sample affixed to a first substrate to a second substrate, in order to facilitate analysis of one or more analytes in situ in the biological sample and/or a matrix on the second substrate.

In some aspects, a specialized adapter having positional markers and/or fiducial markers is applied to the biological sample affixed to the substrate, thus enabling in situ analysis. In some aspects, the biological sample affixed to the incompatible substrate is embedded in a first polymer matrix (e.g., hydrogel matrix) such that the embedded biological sample may be detached and transferred to a second substrate which comprises positional markers and/or fiducial markers and/or a region suitable for in situ analysis and, thus, is compatible with in situ analysis. In some aspects, the embedded biological sample is further embedded in a second polymer matrix and affixed to the second substrate, thus enabling in situ analysis.

In one aspect of the present disclosure, provided herein is a method for processing a biological sample, comprising a) delivering a first matrix-forming material to a biological sample immobilized on a first substrate; b) forming a first three-dimensional polymerized matrix from the first matrix-forming material, thereby embedding the biological sample in the first three-dimensional polymerized matrix; and c) immobilizing the biological sample embedded in the first three-dimensional polymerized matrix to a second substrate. In some embodiments, the biological sample can be a fixed sample. In some embodiments, the biological sample can be a sample embedded in a matrix (e.g., an FFPE sample) prior to the delivering of the first matrix-forming material to the sample. The first substrate can be any substrate, such as plastic or glass slides, and the second substrate can be a specialized substrate for in situ analysis.

In one aspect of the present disclosure, provided herein is a method for processing a biological sample, comprising a) delivering a first matrix-forming material to a biological sample immobilized on a first substrate; b) forming a first three-dimensional polymerized matrix from the first matrix-forming material, thereby embedding the biological sample in the first three-dimensional polymerized matrix; c) immobilizing the biological sample embedded in the first three-dimensional polymerized matrix to a second substrate; d) delivering a second matrix-forming material to the biological sample immobilized on the second substrate; and e) forming a second three-dimensional polymerized matrix from the second matrix-forming material, thereby embedding the biological sample in the second three-dimensional polymerized matrix.

In any of the preceding embodiments, the method can further comprise fixing the biological sample prior to the delivering step in a). In any of the preceding embodiments, the method can further comprise permeabilizing the biological sample prior to the delivering step in a). In any of the preceding embodiments, a plurality of molecules in the biological sample can be attached to the first three-dimensional polymerized matrix to substantially retain the relative three-dimensional spatial relationship among the molecules.

In any of the preceding embodiments, the method can further comprise clearing the biological sample embedded in the first three-dimensional polymerized matrix prior to the immobilizing step in c). In any of the preceding embodiments, the clearing step can comprise contacting the biological sample with a digestion enzyme. In some embodiments, the digestion enzyme can be proteinase K. In any of the preceding embodiments, the method can further comprise detaching the biological sample embedded in the first three-dimensional polymerized matrix from the first substrate prior to the immobilizing step in c).

In any of the preceding embodiments, the method can further comprise expanding the first three-dimensional polymerized matrix, wherein the expansion facilitates detachment of the biological sample from the first substrate. In any of the preceding embodiments, the detaching step can comprise contacting the biological sample embedded in the first three-dimensional polymerized matrix with a separation reagent prior to the immobilizing step in c). In any of the preceding embodiments, the separation reagent can comprise a detergent, a salt, a digestion enzyme, a protein denaturant, or any combination thereof. In some embodiments, the digestion enzyme can be a proteinase such as proteinase K. In any of the preceding embodiments, the detaching step can comprise immersing the biological sample in a separation reagent. In any of the preceding embodiments, the detaching step can comprise applying a shear stress to the biological sample. In any of the preceding embodiments, the detaching step can comprise flowing a separation reagent through the first three-dimensional polymerized matrix. In any of the preceding embodiments, the detaching step can comprise sonicating, shaking, and/or agitating the biological sample in the presence of a separation reagent. In any of the preceding embodiments, the detaching step can comprise immersing the biological sample in a separation reagent; applying a shear stress to the biological sample; flowing a separation reagent through the first three-dimensional polymerized matrix; and/or sonicating, shaking, and/or agitating the biological sample in the presence of the separation reagent, in any combination. In any of the preceding embodiments, the detaching step and clearing the biological sample can be performed in a single step. In some embodiments, the single step can comprise contacting the biological sample with a separation reagent comprising a proteinase. In some embodiments, the proteinase can be proteinase K.

In any of the preceding embodiments, the method can further comprise transferring the detached biological sample to the second substrate prior to the immobilizing step in c). In any of the preceding embodiments, a plurality of molecules in the biological sample can be attached to the first and/or second three-dimensional polymerized matrix to substantially retain the relative three-dimensional spatial relationship among the molecules. In any of the preceding embodiments, a surface of the second substrate can comprise a coating that includes one or more substances to facilitate attachment of a biological sample (e.g., tissue sample) and/or a three-dimensional polymerized matrix. In any of the preceding embodiments, a surface of the second substrate can be functionalized with one or more substances to facilitate attachment of a biological sample (e.g., tissue sample) and/or a three-dimensional polymerized matrix. In any of the preceding embodiments, the one or more substances to facilitate attachment to the surface of the second substrate can comprise lectins, poly-lysine, antibodies, polysaccharides, or binding moieties capable of covalent binding, or any combination thereof. In any of the preceding embodiments, the one or more substances can comprise acryloyls.

In any of the preceding embodiments, a surface of the second substrate can further comprise a coating that includes one or more substances to deter attachment. In any of the preceding embodiments, a surface of the second substrate can be functionalized with one or more substances to deter attachment. In some embodiments, the one or more substances to facilitate attachment and the one or more substances to deter attachment form a pattern on the second substrate. In any of the preceding embodiments, the surface of the second substrate is patterned (e.g., with the one or more substances to facilitate attachment and the one or more substances to deter attachment) to provide an adhesive region and a non-adhesive region on the surface of the second substrate. In some embodiments, the adhesive region is separated from an adjacent adhesive region by one or more non-adhesive regions.

In any of the preceding embodiments, a surface of the second substrate can have a recessed cavity. In any of the preceding embodiments, the recessed cavity can comprise a coating or can be functionalized with one or more substances to facilitate attachment of a biological sample (e.g., tissue sample) and/or a three-dimensional polymerized matrix. In any of the preceding embodiments, the biological sample can be a tissue slice between about 1 μm and about 50 μm in thickness. In some embodiments, the tissue slice can be between about 5 μm and about 35 μm in thickness. In any of the preceding embodiments, the first three-dimensional polymerized matrix and the second three-dimensional polymerized matrix can be formed by subjecting the first matrix-forming material and second matrix-forming material to polymerization. In any of the preceding embodiments, the polymerization can be initiated by adding a polymerization-inducing catalyst, UV light or functional cross-linkers. In any of the preceding embodiments, the first matrix-forming material and/or second matrix-forming material can comprise polyacrylamide, cellulose, alginate, polyamide, cross-linked agarose, cross-linked dextran or cross-linked polyethylene glycol. In any of the preceding embodiments, the first matrix-forming material can comprise a plurality of stimulus-responsive matrix-forming monomers and wherein the first three-dimensional polymerized matrix expands when exposed to a suitable stimulus. In some embodiments, the stimulus-responsive matrix-forming monomers can comprise a sodium acrylate monomer and/or an N-isopropylacrylamide/acrylamide monomer. In any of the preceding embodiments, the plurality of stimulus-responsive matrix-forming monomers can comprise acrylic acid and acrylamide monomers, or N-isopropylacrylamide and acrylamide monomers.

In any of the preceding embodiments, the method can further comprise expanding the biological sample embedded in the first three-dimensional polymerized matrix prior to the immobilizing step. In any of the preceding embodiments, the expanding step can comprise subjecting the first three-dimensional polymerized matrix to a change in salt concentration and/or a temperature change. In any of the preceding embodiments, the first matrix-forming material can comprise acrylamide (AM) and N,N-methylenebisacrylamide (BIS). In any of the preceding embodiments, the first matrix-forming material can comprise acrylamide (AM), polydopamine (PDA) and N,N′-diallyltartardiamide (DATD).

In any of the preceding embodiments, the first matrix-forming material can comprise a plurality of matrix-forming monomers comprising functional groups capable of forming covalent bonds with one or more molecules in the biological sample. In any of the preceding embodiments, the method can further comprise forming covalent bonds between the biological sample and the first three-dimensional polymerized matrix in which the biological sample is embedded. In any of the preceding embodiments, the second matrix-forming material can comprise one or more cross-linking agents or a plurality of matrix-forming monomers capable of forming cross-linking bonds. In any of the preceding embodiments, the one or more cross-linking agents can comprise N,N,N′,N′-tetramethylethylenediamine (TEMED), N,N-methylenebisacrylamide (BIS), or N,N′-diallyltartardiamide (DATD), or any combination thereof. In any of the preceding embodiments, the second matrix-forming material can further comprise one or more initiators. In some embodiments, the one or more initiators can comprise potassium peroxodisulfate and/or ammonium sulfate. In any of the preceding embodiments, the plurality of matrix-forming monomers capable of forming cross-linking bonds can comprise matrix-forming monomers comprising one or more Click-compatible moieties. In some embodiments, the Click-compatible moieties can be azido and/or alkynyl moieties. In any of the preceding embodiments, one or more substances that facilitate sample attachment can be present on the second substrate and/or in the second three-dimensional polymerized matrix.

In any of the preceding embodiments, the method can further comprise sandwiching the biological sample embedded in the first three-dimensional polymerized matrix between the first substrate and the second substrate; and removing the first substrate.

In any of the preceding embodiments, the first delivering step a) can comprise delivering the first matrix-forming material to a first space between a surface of the first substrate and a surface of the second substrate, wherein: the first space is at least partially enclosed by a first spacer between the first and second substrates, the first space contains the biological sample immobilized on the surface of the first substrate, and the first three-dimensional polymerized matrix formed in step b) is sandwiched between the first substrate and the second substrate. In any of the preceding embodiments, the first spacer can be non-integral to the first or second substrate. In any of the preceding embodiments, the second delivering step d) can comprise delivering the second matrix-forming material to a second space between a surface of the second substrate and a surface of a third substrate, wherein the second space is at least partially enclosed by a second spacer between the second and third substrates, and the second space contains the biological sample embedded in the first three-dimensional polymerized matrix.

In any of the preceding embodiments, the second spacer can be non-integral to the second or third substrate. In any of the preceding embodiments, the first spacer and/or second spacer can be an adhesive tape. In some embodiments, the adhesive tape can have a thickness of between about 5 μm and about 100 μm. In some embodiments, the adhesive tape can have a thickness of between about 10 μm and about 20 μm. In any of the preceding embodiments, the first spacer and/or second spacer can comprise a plurality of microparticles. In some embodiments, the average diameter of the microparticles can be between about 10 μm and 50 μm. In any of the preceding embodiments, the method can further comprise analyzing one or more analytes in the biological sample embedded in the first three-dimensional polymerized matrix and/or the second three-dimensional polymerized matrix in situ on the second substrate. Also disclosed is a biological sample for in situ analysis obtained according to any of the methods described herein.

In another aspect, provided herein is a biological sample for in situ analysis, comprising a biological sample; a first three-dimensional polymerized matrix; a second three-dimensional polymerized matrix; and a substrate, wherein the biological sample is embedded in the first three-dimensional polymerized matrix, wherein the first three-dimensional polymerized matrix is further embedded in the second three-dimensional polymerized matrix, and wherein the second three-dimensional polymerized matrix is immobilized to the substrate. In yet another aspect, provided herein is a biological sample for in situ analysis obtained according to the methods of the present disclosure, wherein the biological sample is embedded in a first three-dimensional polymerized matrix, wherein the first three-dimensional polymerized matrix is further embedded in a second three-dimensional polymerized matrix, and wherein the second three-dimensional polymerized matrix is immobilized to a substrate. In any of the preceding embodiments, the substrate can comprise one or more positioning markers and/or fiducial markers on a same surface to which the second three-dimensional polymerized matrix is immobilized.

In another aspect, provided herein is a kit, comprising a first matrix-forming material; a second matrix-forming material; a substrate, wherein: the substrate comprises a coating with or is functionalized with one or more substances to facilitate attachment of a biological sample and/or a three-dimensional polymerized matrix, and the substrate comprises one or more positioning markers and/or fiducial markers. In some embodiments, the kit further comprises instructions for use of the kit components. In any of the preceding embodiments, the one or more substances to facilitate attachment can comprise lectins, poly-lysine, antibodies, polysaccharides, or binding moieties capable of covalent binding. In some embodiments, the one or more substances to facilitate attachment can comprise acryloyls. In any of the preceding embodiments, the substrate can further comprise a coating with or can be functionalized with one or more substances (e.g., one or more hydrophobic substances) to deter attachment, and wherein the one or more substances to facilitate attachment and the one or more substances to deter attachment are patterned to provide an adhesive region and a non-adhesive region on the surface of the substrate. In any of the preceding embodiments, the kit can further comprise one or more separation agents. In any of the preceding embodiments, the one or more separation agents can comprise a detergent, a salt, a digestion enzyme (such as a proteinase, e.g., proteinase K), or a protein denaturant. In any of the preceding embodiments, the first matrix-forming material can comprise a plurality of stimulus-responsive matrix-forming monomers. In any of the preceding embodiments, the kit can further comprise one or more cross-linking agents.

In one aspect, provided herein is an adapter for a biological sample affixed to a substrate for in situ analysis, comprising: a body having a first surface, a second surface, and at least one hole extending through the body from the first surface to the second surface; wherein the first surface comprises one or more positioning markers and/or fiducial markers and wherein the second surface is configured to contact and to be supported by a substrate, wherein the at least one hole is configured in the body to be positioned over the substrate to form at least one sample well, wherein at least one biological sample or a portion thereof is affixed to the substrate and is contained within a sample well.

In any of the preceding embodiments, the adapter comprises a planar body and the second surface is configured to contact and be supported by a planar substrate. In some embodiments, the planar substrate can be a glass slide. In any of the preceding embodiments, the body of the adapter has a thickness of between about 1 μm and 200 μm. In some embodiments, the body of the adapter has a thickness of about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, or 200 μm. In any of the preceding embodiments, the body of the adapter can have a thickness greater than or equal to the thickness of the at least one biological sample. In some embodiments, the at least one biological sample can have a thickness between 1 μm and 50 μm or between 5 μm and 20 μm. In any of the preceding embodiments, the outer perimeter of the body of the adapter can be substantially the same as the outer perimeter of the substrate. In any of the preceding embodiments, the first surface of the adapter can comprise a coating with or can be functionalized with one or more substances to facilitate attachment of a biological sample (e.g., tissue sample) and/or a three-dimensional polymerized matrix. In any of the preceding embodiments, the at least one hole can comprise inner walls, and the inner walls of the at least one hole can comprise a coating with, or can be functionalized with, one or more substances to facilitate attachment of a biological sample (e.g., tissue sample) and/or a three-dimensional polymerized matrix. In any of the preceding embodiments, the one or more substances to facilitate attachment can comprise lectins, poly-lysine, antibodies, polysaccharides, or binding moieties capable of covalent binding, or any combination thereof. In some embodiments, the one or more substances can comprise acryloyls. In any of the preceding embodiments, the first surface and/or the inner walls of the at least one hole of the adapter can comprise a coating with, or can be functionalized with, one or more substances to deter attachment of one or more analytes, a biological sample (e.g., tissue sample) and/or a three-dimensional polymerized matrix. In any of the preceding embodiments, the second surface can comprise a coating or can be functionalized with one or more substances to facilitate attachment to a biological sample (e.g., tissue sample) and/or a three-dimensional polymerized matrix. In any of the preceding embodiments, the adapter can be made of glass, an elastomer, or adhesive tape. In some embodiments the elastomer is polydimethylsiloxane (PDMS).

In another aspect, provided herein is a method of processing a biological sample, comprising a) applying an adapter to a substrate, wherein a biological sample is affixed to the substrate, wherein the adapter comprises a body having a first surface, a second surface, and a first hole extending through the body from the first surface to the second surface; wherein the first surface comprises one or more positioning markers and/or fiducial markers, wherein the second surface is in contact with the substrate, wherein the first hole is configured to be positioned over the substrate to form a sample well, wherein the biological sample is contained within the sample well; b) delivering a matrix-forming material to the sample well containing the biological sample; and c) forming a three-dimensional polymerized matrix from the matrix-forming material, thereby embedding the biological sample in the three-dimensional polymerized matrix.

In any of the preceding embodiments, the substrate to which the biological sample is affixed may not have positioning markers and/or fiducial markers. In any of the preceding embodiments, the adapter can comprise a planar body and the second surface can be configured to contact and be supported by a planar substrate. In some embodiments, the planar substrate can be a glass slide. In any of the preceding embodiments, the body of the adapter can have a thickness of between about 1 μm and 200 μm. In some embodiments, the body of the adapter has a thickness of about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, or 200 μm. In any of the preceding embodiments, the body of the adapter can have a thickness greater than or equal to the thickness of the biological sample. In some embodiments, the biological sample can have a thickness between 1 μm and 50 μm or between 5 μm and 20 μm. In any of the preceding embodiments, the outer perimeter of the body of the adapter can be substantially the same as the outer perimeter of the substrate.

In any of the preceding embodiments, the method can further comprise applying a cover to the first surface of the adapter, prior to step b) or after step c), wherein the cover comprises a second body, wherein the second body comprises a third surface, a fourth surface, and a second hole extending through the second body from the third surface to the fourth surface, and wherein the biological sample and the first hole are positioned centrally under the second hole and the second hole has a cross-sectional area such that the biological sample, the first hole, the one or more positioning markers and/or fiducial markers of the first surface are visible through the second hole. In any of the preceding embodiments, the outer perimeter of the cover can be substantially the same as the body of the adapter. In any of the preceding embodiments, the first surface of the adapter can comprise a coating with, or can be functionalized with, one or more substances to facilitate attachment of a biological sample (e.g., tissue sample) and/or a three-dimensional polymerized matrix. In any of the preceding embodiments, the first hole can comprise inner walls, and the inner walls of the first hole can comprise a coating with, or can be functionalized with, one or more substances to facilitate attachment of a biological sample (e.g., tissue sample) and/or a three-dimensional polymerized matrix. In any of the preceding embodiments, the second hole can comprise inner walls, and the inner walls of the second hole can comprise a coating with, or can be functionalized with, one or more substances to facilitate attachment of a three-dimensional polymerized matrix. In any of the preceding embodiments, the one or more substances to facilitate attachment can comprise lectins, poly-lysine, antibodies, polysaccharides, or binding moieties capable of covalent binding. In some embodiments, the one or more substances can comprise acryloyls. In any of the preceding embodiments, the first surface and/or the inner walls of the at least one hole of the adapter can comprise a coating with, or can be functionalized with, one or more substances to deter attachment of one or more analytes, a biological sample (e.g., tissue sample) and/or a three-dimensional polymerized matrix. In any of the preceding embodiments, the adapter can be made of glass, an elastomer, or adhesive tape. In some embodiments, the elastomer can be polydimethylsiloxane (PDMS). In any of the preceding embodiments, the matrix-forming material can comprise a plurality of fluorescent beads, and wherein the thickness of the three-dimensional polymerized matrix can be measured by imaging the fluorescent beads. In some embodiments, the fluorescent beads can have an average diameter of about 0.2 μm.

In any of the preceding embodiments, the three-dimensional polymerized matrix can be formed by subjecting the matrix-forming material to polymerization. In any of the preceding embodiments, the polymerization can be initiated by adding a polymerization-inducing catalyst, UV light or functional cross-linkers. In any of the preceding embodiments, the matrix-forming material can comprise polyacrylamide, cellulose, alginate, polyamide, cross-linked agarose, cross-linked dextran or cross-linked polyethylene glycol. In any of the preceding embodiments, the method can further comprise crosslinking the biological sample embedded in the three-dimensional polymerized matrix. In any of the preceding embodiments, the method can further comprise clearing the biological sample embedded within the three-dimensional polymerized matrix.

In another aspect, provided herein is a sample cassette for in situ analysis, comprising: a biological sample affixed to a substrate; an adapter, comprising a body having a first surface, a second surface, and a first hole extending through the body from the first surface to the second surface, wherein the first surface comprises one or more positioning markers and/or fiducial markers and wherein the second surface is in contact with and supported by the substrate; wherein the first hole is configured in the body to be positioned over the substrate to form a sample well configured to contain the biological sample affixed to the substrate; and a cover, comprising: a second body having a third surface, a fourth surface, and a second hole extending through the second body from the third surface to the fourth surface, wherein one of the third surface and the fourth surface is in contact with and supported by the second surface of the adapter, and wherein the first hole and second hole are configured such that when the cover is placed atop the first surface of the adapter, the first hole is positioned centrally under the second hole and the second hole has a cross-sectional area such that the first hole and the one or more positioning markers and/or fiducial markers of the first surface are visible through the second hole.

In yet another aspect, provided herein is a kit, comprising: an adapter, comprising a body having a first surface, a second surface, and a first hole extending through the body from the first surface to the second surface, wherein the first surface comprises one or more positioning markers and/or fiducial markers and wherein the second surface is configured to contact and to be supported by a substrate, wherein the first hole is configured in the body to be positioned over the substrate to form a sample well configured to contain a biological sample affixed to the substrate; a cover, comprising a second body having a third surface, a fourth surface, and a second hole extending through the second body from the third surface to the fourth surface, wherein the first hole and second hole are configured such that when the cover is placed atop the first surface of the adapter, the first hole is positioned centrally under the second hole and the second hole has a cross-sectional area such that the first hole and the one or more positioning markers and/or fiducial markers of the first surface are visible through the second hole. In some embodiments, the kit further comprises instructions for use of the kit components.

### DESCRIPTION OF THE FIGURES

The present application can be understood by reference to the following description taken in conjunction with the accompanying figures that illustrate certain embodiments of the features and advantages of this disclosure. These embodiments are not intended to limit the scope of the appended claims in any manner.

FIGS. 1A and 1B depict an exemplary specialized substrate 100 for in situ analysis and an exemplary biological sample (e.g., tissue sample) 106 embedded in a three-dimensional polymerized matrix (e.g., a hydrogel matrix) 108 affixed to the specialized substrate. FIG. 1A shows a top view of the specialized substrate 100. In FIG. 1A, one embodiment of the exemplary specialized substrate comprises positional markers and/or fiducial markers 102 which surround an exemplary region 104 of the substrate for in situ analysis. The exemplary region may comprise modifications such as surface functionalization (e.g., to facilitate sample attachment) and/or have specifications (e.g., thickness or other physical, optical or chemical properties) that would facilitate in situ signal detection and/or analysis. The exemplary region may comprise immobilized molecules, such as immobilized oligonucleotides that form a barcoded array, e.g., a spatial array. FIG. 1B shows a cut-out side view in which a biological sample 106 is embedded in a polymer matrix 108 and affixed to the specialized substrate 100 (e.g., glass slide), and a cover 110 having an open hole 112 is positioned such that the biological sample and positional markers and/or fiducial markers of the specialized substrate are visible and accessible through the open hole of the cover for in situ analysis. Matrix-forming material may be delivered to the open hole and polymerized to embed the biological sample in a three-dimensional polymerized matrix.

FIGS. 2A and 2B depict exemplary processes 200 and 222 according to the present disclosure for processing a biological sample for in situ analysis, shown from cut-out side views for each step.

FIG. 2A shows in one embodiment of the methods provided herein, a process 200, whereby a biological sample 202 immobilized to a non-specialized first substrate 204 (e.g., non-specialized for in situ analysis of the biological sample) is transferred to a second substrate, which is a specialized substrate 212 (e.g., specialized for in situ analysis of the biological sample) having positional markers and/or fiducial markers 214 suited for in situ analysis. In step 206, the biological sample affixed to the non-specialized first substrate is embedded in a first three-dimensional polymerized matrix 208. In step 210, the embedded biological sample is then transferred from the first substrate to the specialized second substrate 212 having positional markers and/or fiducial markers 214 and/or a region 216 (e.g., a region for attachment of a sample for in situ analysis). In step 218, the embedded biological sample is further embedded in a second three-dimensional polymerized matrix 220, and affixed to the second substrate 212, thereby providing a biological sample for in situ analysis.

FIG. 2B shows an alternative process 222, which is a variation of the process 200, employing a sandwich configuration of the non-specialized first substrate 204, a specialized second substrate 212, as well as the use of spacers 226 and 236 and a third substrate 238 to control the thickness of the two hydrogel matrices formed. As shown in FIG. 2B, step 224, a biological sample 202 immobilized to a non-specialized first substrate 204 is sandwiched between the non-specialized first substrate and a specialized second substrate 212. A first set of spacers 226, such as adhesive tape or microparticles, are optionally used to control the distance between the two substrates. In step 228, a first-matrix-forming material is delivered to the space formed by the sandwich of the two substrates and the spacers and polymerized to provide a first three-dimensional polymerized matrix 230. The sandwich configuration may be inverted such that the embedded biological sample is supported by the specialized second substrate. In step 232, the embedded sample is detached from the non-specialized substrate, e.g., by expanding the first three-dimensional polymerized matrix and/or applying a separation agent to the non-specialized substrate. The non-specialized substrate may be removed during or after the detaching step. In step 234, a third substrate 238 and second set of spacers 236 are applied to the embedded sample and specialized substrate. In step 240, a second-matrix forming material is delivered to the space formed by the sandwich of the second and third substrates and the second set of spacers and polymerized to further embed the biological sample and first three-dimensional polymerized matrix in a second three-dimensional polymerized matrix 242. The sample is immobilized to the specialized substrate and the third substrate removed, thereby providing a biological sample for in situ analysis.

FIGS. 3A-3F depict an exemplary adapter 300 according to one embodiment of the present disclosure, a cover 306, a biological sample 312 affixed to a unmarked, non-specialized substrate (e.g., glass slide) 310, and the combined configuration and assembly of the adapter, cover and biological sample on the unmarked, non-specialized substrate for use in situ analysis. FIG. 3A depicts a top view of the adapter 300 having a first hole 302 and positional markers and/or fiducial markers 304 located around the hole. The adapter may also have a coating or surface functionalization, not shown, to facilitate or deter attachment of a biological sample, a three-dimensional matrix, and/or any analytes. FIG. 3B depicts a top view of a cover 306 for the adapter, having a second hole 308. As shown in FIG. 3C (cut-out side view), the adapter 300 and cover 306 are configured such that the cover 306 may be overlaid on the adapter 300 and the second hole 308 is sufficiently large to allow access to the first hole 302 and allows visualization the positional markers and/or fiducial markers 304 on the adapter 300.

FIGS. 3D-3F further depict a biological sample 312 affixed to an unmarked, non-specialized substrate 310 configured with the adapter 300 and cover 306. In FIG. 3D (cut-out side view), the first hole 302 of the adapter 300 is configured such that the biological sample 312 is fully contained within a sample well formed by the first hole 302 of the adapter 300 and the underlying non-specialized substrate 310. As illustrated in FIG. 3E, the cover 306 may be further placed atop the adapter 300 and is configured such that the biological sample 312 and sample well remain accessible and visible through (unobstructed by) the second hole 308 of the cover 306, once assembled. FIGS. 3E and 3F show a cut-out side view (FIG. 3E) and a top view (FIG. 3F) of the assembled sample cassette (cover 306, adapter 300, biological sample 312, and non-specialized substrate 310) for in situ analysis.

FIG. 4 depicts an exemplary process 400 according to the present disclosure for preparing a biological sample for in situ analysis, shown from a cut-out side view for each step. In one embodiment of the methods provided herein, a biological sample 402 immobilized to an unmarked substrate 404 is fitted with an adapter 408 having positional markers and/or fiducial markers 410 and a cover 416. In step 406, a biological sample 402 affixed to an unmarked substrate 404 is fitted with an adapter 408 having positional markers and/or fiducial markers 410. The adapter 408 is configured to have a first hole 412 which combines with the unmarked substrate 404 to form a sample well containing the biological sample 402. In step 414, a cover 416 having a second hole 418 is placed atop the adapter. The cover is configured such that the second hole 418 is centered over the first hole and biological sample (and the sample well formed by the first hole and substrate), further such that the biological sample, sample well, and positional markers and/or fiducial markers are accessible and visible through the second hole. In step 420, matrix-forming material is added to the sample well, and optionally also the second hole, and polymerized, thereby embedding the biological sample in a three-dimensional polymerized matrix 422.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

Provided herein, in some aspects, are methods and devices for preparing biological samples for processing and in situ analysis. In some embodiments, the methods for preparing the thin three-dimensional polymerized matrices are methods for preparing hydrogel-embedded biological samples.

In certain embodiments, the hydrogel-embedded biological samples obtained according to the methods provided herein may further be used in or for methods for detecting the presence of and/or quantifying analytes, such as nucleic acids, in cells, tissues, organs or organisms. In some embodiments, the present disclosure provides methods for high-throughput profiling of a large number of targets in situ, including information about the spatial distribution of such targets (e.g., RNA transcripts and/or DNA loci) in a tissue sample.

The section headings used herein are for organizational purposes only and are not to be construed as limiting the subject matter described.

### I. Overview

Prior to the imaging of biological samples, such as tissue samples or layers of cells, the biological samples are typically processed and affixed to substrates, e.g., microscopy slides, and embedded in three-dimensional polymerized matrices, such as hydrogel matrices, to prevent detachment from the microscopy slides, to preserve information about the relative spatial distribution of molecules within the tissue (e.g., by linking a molecule of interest to its original location and detecting co-localization with other molecules of interest), and to mitigate sample degradation. The fixation of biological samples, such as tissue sections, to substrates, such as microscopy slides, and subsequent embedding of the biological samples in three-dimensional polymerized matrices, e.g., hydrogel matrices, are key steps in sample preparation for stabilizing the integrity of biological samples and enabling further treatment of the biological sample to facilitate analysis, such as in situ analysis. For example, after being affixed to a suitable substrate, such as a glass slide, and being embedding in a polymerized matrix, such as a hydrogel matrix, a biological sample may be subjected to any number of post-processing steps including, but not limited to, clearing, cross-linking, expansion, etc., with reduced risk of damage to and decreased structural instability of the biological sample.

In some circumstances, specialized substrates suited for a specific analytical technique to be performed may be used. For example, in situ analysis such as transcriptional profiling may involve detecting analytes present at low concentrations, such as nucleic acids, and may rely upon highly sensitive measurements, for example, single molecule fluorescence signals. As such, in situ analysis may use specialized substrates that are critical to obtaining a measurement. Specialized substrates may, for example, be coated with oligonucleotide arrays which may bind to RNA present in the biological samples and/or may be modified with positional markers and/or fiducial markers in order to allow for positional mapping. These modifications of the substrate not only serve as functional components of the in situ analysis but also facilitate subsequent interpretation. Specialized substrates having these modifications or attributes may include “modified”, “marked” or “in situ” substrates in the present disclosure.

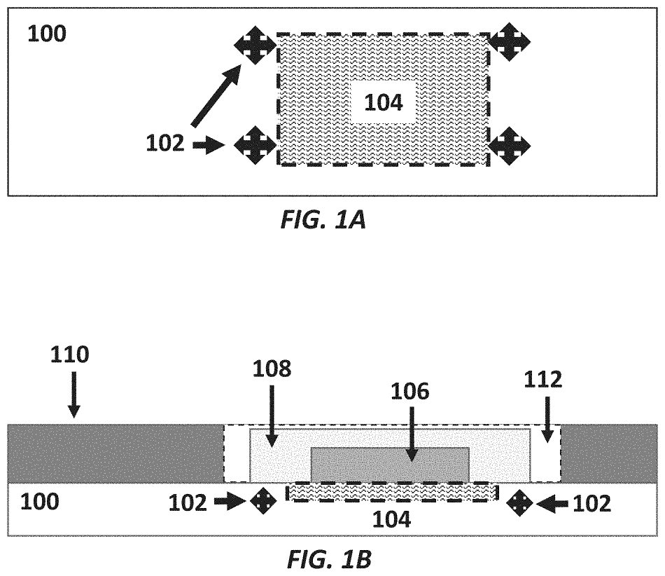

An exemplary specialized substrate for in situ analysis is illustrated in FIG. 1A. As shown in FIG. 1A, the exemplary specialized substrate 100 comprises positional markers and/or fiducial markers 102 and a region 104 onto which a biological sample may be affixed. FIG. 1B further illustrates a fully constructed sample cassette for in situ analysis including the specialized substrate 100, an exemplary biological sample (e.g., tissue sample) 106, and a cover 110. The biological sample 106 is embedded in a three-dimensional polymerized matrix (e.g., a hydrogel matrix) 108, and is immobilized on the specialized slide 100 to the region 104. The body of the cover 110 has a hole 112 large enough to surround the biological sample 106 embedded in the three-dimensional polymerized matrix 108 such that the biological sample 106, the three-dimensional polymerized matrix 108 and the positional markers and/or fiducial markers 102 on the underlying specialized substrate 100 are accessible and visible for imaging.

In some instances, many biological samples (e.g., archived tissue specimens) may not be affixed to specialized substrates, but are instead fixed to substrates that are not specialized (e.g., not specialized for in situ analysis), such as unmodified substrates. Whether out of convenience, or due to intended use with other analytical techniques that do not require the specifications and/or modifications required for in situ analysis, non-specialized substrates (e.g., unmodified plastic or glass slides) may not be compatible with in situ measurements. Thus, biological samples affixed to these non-specialized substrates may not be readily examined by existing in situ analysis methods. As provided herein, such substrates without modifications for in situ analysis may include “unmarked”, “non-specialized”, and/or “unmodified” substrates. Improved methods for sample processing and in situ analysis are needed, particularly for allowing measurement of biological samples affixed to substrates that are not compatible with or not designed for in situ analysis, such as in situ sequencing or in situ hybridization.

The present disclosure addresses this need by providing methods and devices for preparing and processing biological samples for in situ analysis by transferring biological samples from non-specialized substrates (e.g., slides that are not compatible with or modified for in situ analysis) to specialized substrates that are compatible with in situ analysis, or by applying an adapter component to the biological sample and unmodified substrate, wherein the adapter is compatible with in situ analysis.

### II. Sample Transfer Methods

The present disclosure provides methods and devices for processing a biological sample affixed or immobilized to an unmodified substrate, comprising embedding the biological samples in a first three-dimensional polymerized matrix, transferring the embedded biological sample to a second specialized substrate, further embedding the embedded sample in a second three-dimensional polymerized matrix, and immobilizing the second three-dimensional polymerized matrix to the second specialized substrate.

In one aspect of the present disclosure, provided herein are methods that involve the transfer of the biological sample to a modified substrate suitable for in situ analysis. In some embodiments, provided herein are methods that stabilize the biological sample for transfer to a modified substrate, comprising embedding biological samples affixed to unmodified substrates in a first three-dimensional polymerized matrix, detaching the embedded sample from the unmodified substrate, transferring the embedded sample to a modified substrate, and further embedding the biological sample in a second three-dimensional polymerized matrix and affixing the embedded biological sample to the modified substrate.

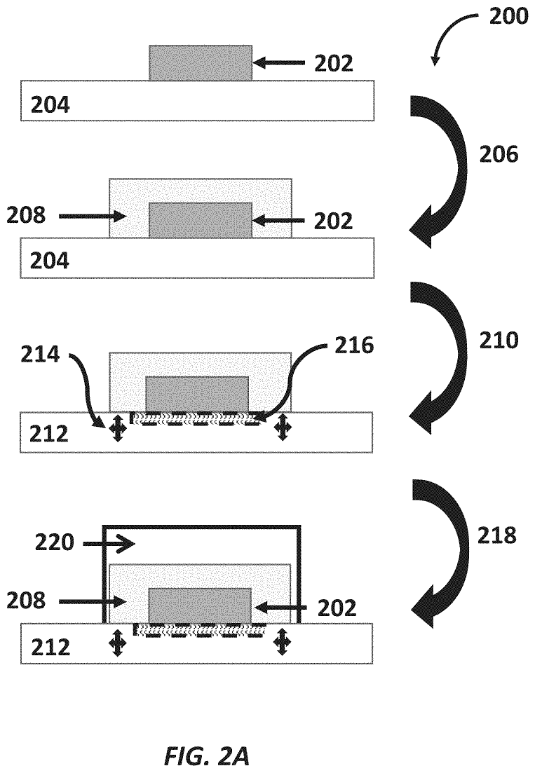

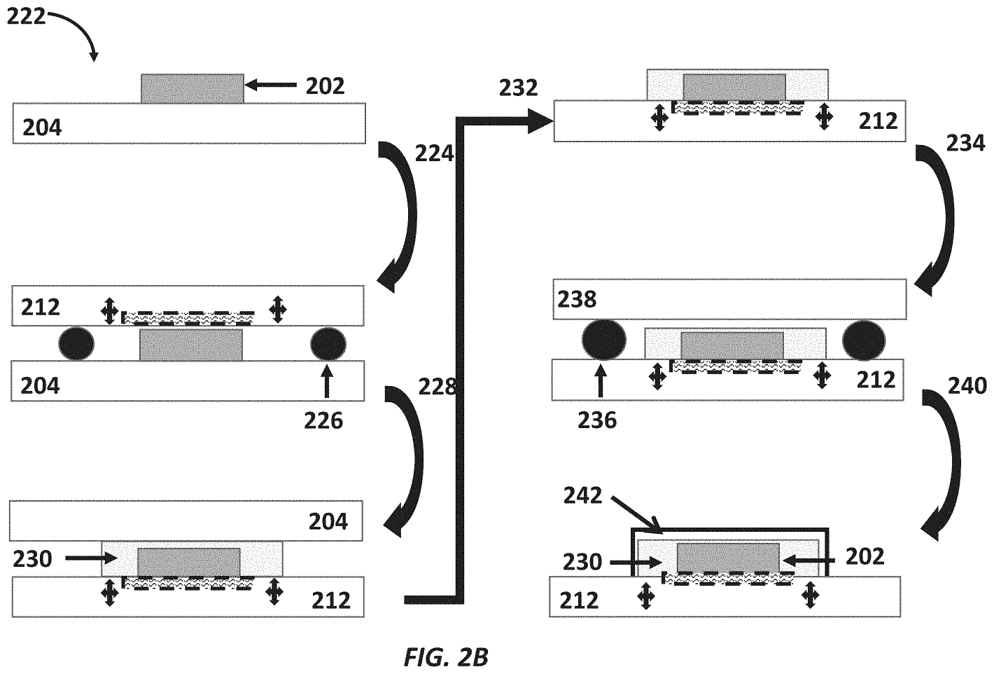

FIG. 2A depicts an exemplary method 200 for the transfer of a biological sample to a specialized substrate. With reference FIG. 2A, at the start of the process 200, a biological sample 202 is already affixed to an unmodified substrate 204. In step 206, a suitable first matrix-forming material (e.g., hydrogel monomers) are added to the biological sample and polymerized so that the biological sample becomes embedded in a first three-dimensional polymerized matrix (e.g., hydrogel matrix) 208. In step 210, the embedded biological sample is detached from the unmodified substrate and transferred to a specialized substrate 212. The detaching step may be achieved, for example, by exposing the embedded biological sample to a suitable separation reagent (such as a detergent, a salt, a digestion enzyme, or a protein denaturant) and/or, if expandable monomers are included in the matrix-forming material, expanding the embedded biological sample to aid detachment. Again, with reference to step 210, the specialized substrate 212 may have one or more modifications that are compatible with in situ analytical techniques, including but not limited to surface functionalization (not shown), positional markers and/or fiducial markers 214 and/or a region 216, such as a region specialized for sample attachment and/or subsequent in situ analysis. In step 218, a second matrix-forming material is delivered to the embedded biological sample (202 and 208) on the specialized substrate 212 and polymerized to form a second three-dimensional polymerized matrix 220. In one embodiment of the sample transfer methods provided herein, at the end of process 200, the biological sample 202 is embedded in a first three-dimensional polymerized matrix 208, which are both further embedded in a second three-dimensional polymerized matrix 220, all of which are affixed to a specialized substrate 212. In some embodiments, immobilization of the biological sample and three-dimensional polymerized matrices may be carried out in the same step as or a separate step from the polymerization of the second three-dimensional polymerized matrix.

FIG. 2B further shows a variation of sample transfer process 222, in which the second specialized substrate 212 is used to sandwich the biological sample 202 affixed to the non-specialized first substrate 204 along with a first set of spacers 226 to control the thickness of the first three-dimensional polymerized matrix formed and to facilitate transfer of the embedded biological sample to the second substrate (e.g., by inverting the sandwich of the pair of substrates). FIG. 2B further illustrates the use of a second set of spacers 236 along with a third substrate 238 to control the thickness of the second three-dimensional polymerized matrix formed.

As shown in FIG. 2B, step 224, a biological sample 202 immobilized to a non-specialized first substrate 204 is sandwiched between the non-specialized first substrate and a specialized second substrate 212. A first set of spacers 226, such as adhesive tape or microparticles, are optionally used to control the distance between the two substrates. In step 228, a first-matrix-forming material is delivered to the space formed by the sandwich of the two substrates and the spacers and polymerized to provide a first three-dimensional polymerized matrix 230. The sandwich configuration may be inverted such that the embedded biological sample is supported by the specialized second substrate. In step 232, the embedded sample is detached from the non-specialized substrate, e.g., by expanding the first three-dimensional polymerized matrix and/or applying a separation agent to the non-specialized substrate. The non-specialized substrate may be removed during or after the detaching step. In step 234, a third substrate 238 and second set of spacers 236 are applied to the embedded sample and specialized substrate. In step 240, a second-matrix forming material is delivered to the space formed by the sandwich of the second and third substrates and the second set of spacers and polymerized to further embed the biological sample and first three-dimensional polymerized matrix in a second three-dimensional polymerized matrix 242. The sample is immobilized to the specialized substrate and the third substrate removed, thereby providing a biological sample for in situ analysis.

In one aspect of the present disclosure, provided herein is a method for processing a biological sample, comprising a) delivering a first matrix-forming material to a biological sample immobilized on a first substrate; b) forming a first three-dimensional polymerized matrix from the first matrix-forming material, thereby embedding the biological sample in the first three-dimensional polymerized matrix; c) immobilizing the biological sample embedded in the first three-dimensional polymerized matrix to a second substrate; d) delivering a second matrix-forming material to the biological sample immobilized on the second substrate; and e) forming a second three-dimensional polymerized matrix from the second matrix-forming material, thereby embedding the biological sample in the second three-dimensional polymerized matrix.

In another aspect, provided herein is a biological sample for in situ analysis, comprising a biological sample; a first three-dimensional polymerized matrix; a second three-dimensional polymerized matrix; and a substrate, wherein the biological sample is embedded in the first three-dimensional polymerized matrix, wherein the first three-dimensional polymerized matrix is further embedded in the second three-dimensional polymerized matrix, and wherein the second three-dimensional polymerized matrix is immobilized to the substrate. In yet another aspect, provided herein is a biological sample for in situ analysis obtained according to the methods of the present disclosure, wherein the biological sample is embedded in a first three-dimensional polymerized matrix, wherein the first three-dimensional polymerized matrix is further embedded in a second three-dimensional polymerized matrix, and wherein the second three-dimensional polymerized matrix is immobilized to a substrate.

In another aspect, provided herein is a kit, comprising a first matrix-forming material; a second matrix-forming material; a substrate, wherein: the substrate comprises a coating with or is functionalized with one or more substances to facilitate attachment of a biological sample and/or a three-dimensional polymerized matrix, and the substrate comprises one or more positioning markers and/or fiducial markers; and optionally, instructions for use thereof.

A. Substrates

For analytical techniques, such as microscopy, planar tissue sections or slices are typically affixed to rigid substrates, such as a glass microscopy slides. The properties and characteristics of the substrates may depend on the desired analytical technique to be performed and/or the biological sample being analyzed. For example, in situ analytical techniques may be carried out on biological samples affixed to specialized substrates having surface functionalization to aid adhesion of the sample, having positional markers and/or fiducial markers for imaging, and/or having an array (e.g., a barcoded oligonucleotide array) to interact with the sample as part of the measurement. In some circumstances, biological samples may be placed on any substrate available at the time or may be archival samples immobilized to substrates incompatible with newer analytical methods. However, it remains difficult to transfer biological samples already affixed to substrates to new substrates, such as specialized substrates for in situ sequencing. The methods provided herein allow for the transfer of biological samples from a first substrate to a second substrate, such as a specialized substrate.

It should be recognized that use of ordinal terms “first” and “second” in the description of the substrates provided herein does not by itself connote any priority, precedence, or order of one substrate over another or the temporal order in which acts of a method are performed, but are used merely as labels to distinguish one substrate having a certain name from another substrate (but for use of the ordinal term) to distinguish the two substrates.

In some embodiments, the first substrate is a glass slide. In some embodiments, the biological sample is affixed to or immobilized on the first substrate.

In some embodiments, the second substrate is a glass slide. In some embodiments, the second three-dimensional polymerized matrix is affixed to or immobilized on the second substrate.

In some embodiments, the first and/or second substrate may comprise a slide or planar component fabricated from any of a variety of suitable materials including, but not limited to, glass (e.g., borosilicate glass, soda lime glass, etc.), fused silica (quartz), silicon, a polymer (e.g., polystyrene (PS), macroporous polystyrene (MPPS), polymethylmethacrylate (PMMA), polycarbonate (PC), cyclic olefin polymer (COP), cyclic olefin copolymer (COC), or any combination thereof.

In further embodiments, the first and/or second substrate may comprise one or more chemical or physical modifications to facilitate the placement of any biological sample embedded on the surface of the first and/or second substrate and its positioning within the three-dimensional polymerized matrix, as well as chemical modifications to facilitate the adhesion of the three-dimensional polymerized matrix to either the first or second substrate. Suitable chemical or physical modifications may include, but are not limited to, coatings comprising one or more substances (which may be adhesive or non-adhesive), functionalization of one or more surfaces with one or more substances (which may be adhesive or non-adhesive), patterned surfaces, etched or roughened surfaces, and one or more recessed cavities.

In some embodiments, a surface of the first substrate comprises a coating comprising, or is functionalized with, one or more substances to facilitate attachment of a biological sample (e.g., tissue sample) and/or a three-dimensional polymerized matrix. In some embodiments, the one or more substances to facilitate attachment to the surface of the first substrate comprise lectins, poly-lysine, antibodies, polysaccharides, or binding moieties capable of covalent binding, optionally wherein the one or more substances comprise acryloyls. In other embodiments, a surface of the first substrate further comprises a coating comprising, or is functionalized with, one or more substances (e.g., one or more hydrophobic substances) to deter attachment, wherein the one or more substances to facilitate attachment and the one or more substances to deter attachment are patterned to provide an adhesive region and a non-adhesive region on the surface of the first substrate.

In some embodiments, a surface of the second substrate comprises a coating comprising, or is functionalized with, one or more substances to facilitate attachment of a biological sample (e.g., tissue sample) and/or a three-dimensional polymerized matrix. In some embodiments, the one or more substances to facilitate attachment to the surface of the second substrate comprise lectins, poly-lysine, antibodies, polysaccharides, or binding moieties capable of covalent binding, optionally wherein the one or more substances comprises acryloyls. In other embodiments, a surface of the second substrate further comprises a coating comprising, or is functionalized with, one or more substances to deter attachment, wherein the one or more substances to facilitate attachment and the one or more substances to deter attachment are patterned to provide an adhesive region and a non-adhesive region on the surface of the second substrate.

Recessed cavities on the first or second substrate may be useful for positioning and/or centering any biological sample intended to be fixed or immobilized therein or to be embedded in the resulting three-dimensional polymerized matrix. In some embodiments, the surface of the first substrate has a recessed cavity. In some embodiments, the biological sample may be fixed or immobilized to the recessed cavity of the first substrate. In some embodiments, the recessed cavity of the first substrate comprises the coating comprising, or is functionalized with, the one or more substances to facilitate attachment. In some embodiments, the surface of the second substrate has a recessed cavity. In some embodiments, the second three-dimensional polymerized matrix may be fixed or immobilized to the recessed cavity of the second substrate. In some embodiments, the recessed cavity of the second substrate comprises the coating comprising, or is functionalized with, the one or more substances to facilitate attachment. In some embodiments, the first and/or second substrate may comprise 1, 2, 3, 4, 5, 6, 7, 8, 9, 10, or more than 10 recessed cavities.

In yet further embodiments, the second substrate may comprise one or more markers, such as a positioning marker or fiducial marker (e.g., a laser inscribed fiducial marker), to facilitate positioning and location of the three-dimensional matrix and/or any biological sample embedded therein in any subsequent measurement and analysis, for example, in microscopy imaging. A suitable positioning marker or fiducial marker may indicate the relative spatial orientation of the biological sample to be evaluated. In some embodiments, the surface of the second substrate comprises one or more positional markers and/or fiducial markers. In some embodiments, the second substrate comprises one or more positioning markers and/or fiducial markers. In some embodiments, the second three-dimensional polymerized matrix is immobilized to the second substrate. In some embodiments, the second substrate comprises one or more positioning markers and/or fiducial markers on the same surface to which the second three-dimensional polymerized matrix is immobilized. In some embodiments, the second substrate may comprise 1, 2, 3, 4, 5, 6, 7, 8, 9, 10, or more than 10 positioning markers and/or fiducial markers.

In some embodiments, the first substrate lacks any surface functionalization or markers. In some embodiments, the surface of the first substrate does not comprise any coating or is not functionalized with one or more substances to facilitate or deter attachment of a biological sample (e.g., tissue sample) and/or a three-dimensional polymerized matrix. In other embodiments, the first substrate does not comprise any positioning markers or fiducial markers. In some embodiments, the first substrate does not comprise any positioning markers or fiducial markers on the surface on which the biological sample is fixed or immobilized.

In some embodiments, a third substrate may be employed with a spacer for the formation of the second three-dimensional polymerized matrix. In some embodiments, the third substrate is a plastic substrate or a glass slide.

B. Transfer Steps

In one aspect, provided herein is a method for processing a biological sample, comprising transferring a biological sample from a non-specialized first substrate to a second specialized substrate for in situ analysis, wherein the transferring step comprises embedding the biological sample in at least two three-dimensional polymerized matrices.

In some embodiments, the method comprises delivering a first matrix-forming material to a biological sample immobilized on a first substrate. In some embodiments, the method further comprises fixing the biological sample prior to delivering the first matrix-forming material to the biological sample immobilized on the first substrate. In some embodiments, the method further comprises fixing the biological sample prior to the delivering step in a). In some embodiments, the method further comprises permeabilizing the biological sample prior to delivering the first matrix-forming material to the biological sample immobilized on the first substrate. In some embodiments, the method further comprises permeabilizing the biological sample prior to the delivering step in a). In other embodiments, the method further comprises permeabilizing the biological sample after fixing the biological sample and prior to delivering the first matrix-forming material to the biological sample immobilized on the first substrate.

In some embodiments, the method further comprises sandwiching the biological sample embedded in the first three-dimensional polymerized matrix between the first substrate and the second substrate; and removing the first substrate.

In some embodiments, the step of delivering the first matrix-forming material comprises delivering the first matrix-forming material to a first space between a surface of the first substrate and a surface of the second substrate, wherein: the first space is at least partially enclosed by a first spacer between the first and second substrates, the first space contains the biological sample immobilized on the surface of the first substrate, and the first three-dimensional polymerized matrix formed therefrom is sandwiched between the first substrate and the second substrate. In some embodiments, the first delivering step a) comprises delivering the first matrix-forming material to a first space between a surface of the first substrate and a surface of the second substrate, wherein: the first space is at least partially enclosed by a first spacer between the first and second substrates, the first space contains the biological sample immobilized on the surface of the first substrate, and the first three-dimensional polymerized matrix formed in step b) is sandwiched between the first substrate and the second substrate. In some embodiments, the first spacer is non-integral to the first or second substrate. In some embodiments, the first spacer is an adhesive tape or ultrathin silicone gasket, optionally wherein the adhesive tape or silicon gasket has a thickness of about 100 μm or between about 10 μm and 20 μm, or comprises a plurality of microparticles, optionally wherein the average diameter of the microparticles is between about 10 μm and 50 μm.

In some embodiments, the method comprises forming a first three-dimensional polymerized matrix from the first matrix-forming material, thereby embedding the biological sample in the first three-dimensional polymerized matrix.

In some embodiments, a plurality of molecules in the biological sample are attached (e.g., adsorbed and/or cross-linked) to the first three-dimensional polymerized matrix to substantially retain the relative three-dimensional spatial relationship among the molecules. In some embodiments, the method further comprises forming covalent and/or non-covalent bonds between the biological sample, or molecules therein, and the first three-dimensional polymerized matrix in which the biological sample is embedded.

In some embodiments, the method comprises immobilizing the biological sample embedded in the first three-dimensional polymerized matrix to a second substrate.

In some embodiments, the method further comprises clearing the biological sample embedded in the first three-dimensional polymerized matrix prior to immobilizing the biological sample embedded in the first three-dimensional polymerized matrix to a second substrate. In some embodiments, the method further comprises clearing the biological sample embedded in the first three-dimensional polymerized matrix prior to the immobilizing step in c). In some instances, the clearing step may comprise the use of a suitable clearing agent, such as an organic solvent, high refractive index aqueous solution, hyperhydrating solution, detergent, digestion enzyme (e.g., proteinase K) or protein denaturant. In some embodiments, the clearing step comprises contacting the biological sample with a digestion enzyme, optionally wherein the digestion enzyme is proteinase K.

In some embodiments, the method further comprises detaching the biological sample embedded in the first three-dimensional polymerized matrix from the first substrate prior to immobilizing the biological sample embedded in the first three-dimensional polymerized matrix to the second substrate. In some embodiments, the method further comprises detaching the biological sample embedded in the first three-dimensional polymerized matrix from the first substrate prior to the immobilizing step in c). In some embodiments, the detaching step may comprise contacting or immersing the biological sample in a separation reagent, applying a shear stress to the biological sample, or sonicating, shaking, and/or agitating the biological sample in the presence of a separation reagent. In some embodiments, the detaching step comprises expanding the first three-dimensional polymerized matrix, wherein the expansion facilitates detachment of the biological sample from the first substrate.

In some embodiments, the method further comprises expanding the biological sample embedded in the first three-dimensional polymerized matrix prior to immobilizing the biological sample embedded in the first three-dimensional polymerized matrix to the second substrate. In some embodiments, the method further comprises expanding the biological sample embedded in the first three-dimensional polymerized matrix prior to the immobilizing step in c). In some embodiments, the expanding step comprises subjecting the first three-dimensional polymerized matrix to a change in salt concentration (e.g., a change of ionic strength) or a temperature change.

In some embodiments, the detaching step comprises contacting the biological sample embedded in the first three-dimensional polymerized matrix with a separation reagent prior to immobilizing the biological sample embedded in the first three-dimensional polymerized matrix to the second substrate. In some embodiments, the detaching step comprises contacting the biological sample embedded in the first three-dimensional polymerized matrix with a separation reagent prior to the immobilizing step in c). In some embodiments, the separation reagent comprises a detergent, a salt, a digestion enzyme, a protein denaturant, or any combination thereof, optionally wherein the digestion enzyme is a proteinase.

In some embodiments, the detaching step comprises immersing the biological sample in a separation reagent; applying a shear stress to the biological sample; flowing a separation reagent through the first three-dimensional polymerized matrix; sonicating, shaking, or agitating the biological sample in the presence of the separation reagent, or any combination thereof.

In some embodiments, the detaching step and clearing the biological sample are performed in a single step, optionally wherein the single step comprises contacting the biological sample with a separation reagent comprising a proteinase.

In some embodiments, the method further comprises transferring the detached biological sample to the second substrate prior to immobilizing the biological sample embedded in the first three-dimensional polymerized matrix to the second substrate. In some embodiments, the method further comprises transferring the detached biological sample to the second substrate prior to the immobilizing step in c).

In some embodiments, the method comprises delivering a second matrix-forming material to the biological sample immobilized on the second substrate.

In some embodiments, the step of delivering the second matrix-forming material to the biological sample immobilized on the second substrate comprises delivering the second matrix-forming material to a second space between a surface of the second substrate and a surface of a third substrate, the second space is at least partially enclosed by a second spacer between the second and third substrates, the second space contains the biological sample embedded in the first three-dimensional polymerized matrix. In some embodiments, the second delivering step d) comprises delivering the second matrix-forming material to a second space between a surface of the second substrate and a surface of a third substrate, the second space is at least partially enclosed by a second spacer between the second and third substrates, the second space contains the biological sample embedded in the first three-dimensional polymerized matrix. In some embodiments, the second spacer is non-integral to the second or third substrate. In some embodiments, the second spacer is an adhesive tape or ultrathin silicone gasket, optionally wherein the adhesive tape or silicon gasket has a thickness of about 100 μm or between about 10 μm and 20 μm, or comprises a plurality of microparticles, optionally wherein the average diameter of the microparticles is between about 10 μm and 50 μm.

In some embodiments, the method comprises forming a second three-dimensional polymerized matrix from the second matrix-forming material, thereby embedding the biological sample in the second three-dimensional polymerized matrix.

In some embodiments, a plurality of molecules in the biological sample are attached (e.g., adsorbed or cross-linked) to the second three-dimensional polymerized matrix to substantially retain the relative three-dimensional spatial relationship among the molecules. In some embodiments, one or more substances that facilitate covalent or non-covalent sample attachment are present on the second substrate and/or in the second three-dimensional polymerized matrix.

In some embodiments, the method further comprises analyzing one or more analytes (e.g., detecting and/or quantifying the presence of one or more analytes) in the biological sample embedded in the first three-dimensional polymerized matrix and/or the second three-dimensional polymerized matrix in situ on the second substrate.

C. Biological Samples for In Situ Analysis

The present disclosure also provides biological samples for in situ analysis that may be obtained from or produced by the methods described herein. In one aspect, the biological samples obtained by the sample transfer methods may comprise a biological sample embedded in at least two three-dimensional polymerized matrices, which may have the same or different compositions.

In one aspect, provided herein is a biological sample for in situ analysis obtained according the sample transfer methods of the present disclosure. In another aspect, provided herein is a biological sample for in situ analysis, comprising a biological sample; a first three-dimensional polymerized matrix; a second three-dimensional polymerized matrix; and a substrate, wherein the biological sample is embedded in the first three-dimensional polymerized matrix, wherein the first three-dimensional polymerized matrix is further embedded in the second three-dimensional polymerized matrix, and wherein the second three-dimensional polymerized matrix is immobilized to the substrate. In some embodiments, the substrate comprises one or more positioning markers and/or fiducial markers on the same surface to which the second three-dimensional polymerized matrix is immobilized.

In still another aspect, provided herein are uses of and methods of using the biological samples for in situ analysis provided herein. In some embodiments, provided herein are methods of analyzing one or more analytes (e.g., detecting and/or quantifying the presence of one or more analytes) in the biological sample embedded in the first three-dimensional polymerized matrix and/or the second three-dimensional polymerized matrix in situ on the substrate.

### III. Sample Adapter Methods

The present disclosure also provides methods and devices (e.g., specialized substrates, adapters, and covers) for processing a biological sample affixed or immobilized to an unmodified substrate, comprising applying an adapter to the biological sample in a first three-dimensional polymerized matrix, wherein the adapter comprises one or more elements, such as surface functionalization, one or more positioning markers and/or fiducial markers, and/or probe arrays (e.g., barcoded oligonucleotide arrays), to make the sample compatible with in situ analysis and/or to facilitate in situ analysis. In some embodiments, the methods provided herein further comprise applying a cover to the adapter and/or forming a three-dimensional polymerized matrix to embed the biological sample. The adapter and the cover are specially configured for their respective holes to accommodate the shape, size and location of the biological sample on the initial unmodified slide. In some embodiments, the adapter and cover are further configured to have the same profile (outer perimeter or footprint) as the initial unmodified slide.

In other aspects of the present disclosure, provided herein are methods that involve the application of an adapter to the biological sample affixed to an unmodified substrate, which makes the biological sample affixed to the unmodified substrate compatible with in situ analytical methods, and adapters therefor. In one aspect, provided herein are methods that utilize an adapter, wherein the adapter comprises one or more modifications compatible with in situ analytical methods, such as positional markers and/or fiducial markers and/or probe arrays. In still yet another aspect of the present disclosure, provided herein is an adapter comprising one or more modifications compatible with in situ analytical methods, such as positional markers and/or fiducial markers and/or probe arrays.

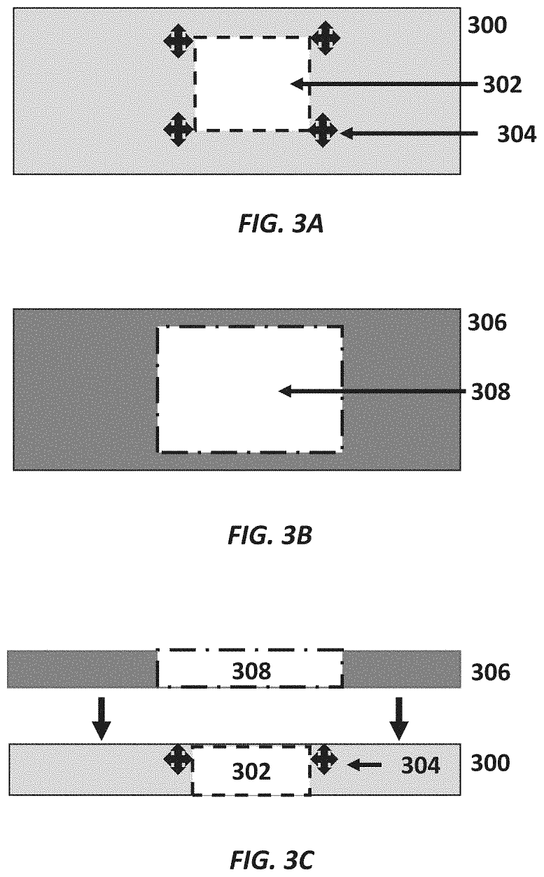

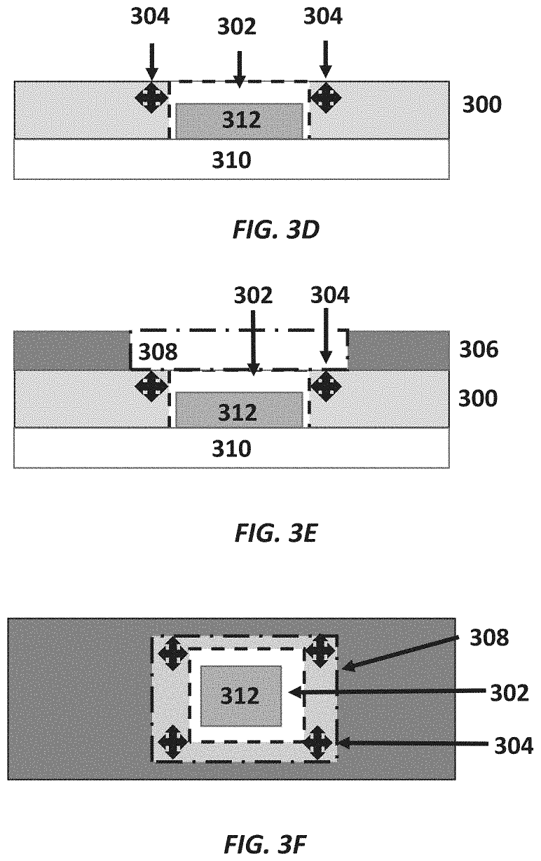

FIG. 3A-3F depict an exemplary adapter according the present disclosure, along with the biological sample affixed to the unmodified substrate and a cover. FIG. 3A, depicts a top view of a planar adapter 300 according to one embodiment of the present disclosure. The adapter 300 comprises a hole 302 extending through the body of the planar adapter, and one or more positional markers and/or fiducial markers 304 on one surface of the adapter. The hole 302 of the adapter is configured such that the hole is sufficiently larger in area than the biological sample to be processed that the biological sample may sit completely within the void space of the hole, optionally without contacting the inner walls of the hole. In some instances, the surfaces and inner walls of the adapter may further comprise surface functionalization (not shown). FIG. 3B depicts a top view of a cover 306 for the adapter 300, wherein the cover has a hole 308 extending through the body of the cover. FIG. 3C depicts a cut-out side view of both the adapter 300 and cover 306. As shown in FIG. 3C, the cover is placed atop the surface of the adapter having the positional markers and/or fiducial markers 304. As further shown in FIGS. 3A-3C, in one embodiment of the adapter and cover, the hole 308 of the cover 306 is centered over the hole 302 of the adapter 300, and is sufficiently larger in area than the hole 302 of the adapter that neither the positional markers and/or fiducial markers of the adapter, nor any biological sample siting within hole 302 of the adapter, is obstructed from view by the body of the cover 306.

FIG. 3D further illustrates a cut-out side view of the assembly of an exemplary adapter and a biological sample affixed to an unmodified substrate. In FIG. 3D, a biological sample 312 is affixed to an unmodified substrate 310. An exemplary adapter 300 is placed atop the unmodified substrate, with any positional markers and/or fiducial markers 304 facing upward and away from the surface of the unmodified substrate to which the biological sample is affixed. The hole 302 of the adapter forms a sample well with the unmodified substrate 310, such that the biological sample 312 rests within and is fully contained by the sample well. FIGS. 3E and 3F further show a cut-out side view and a top view of a fully assembled sample cassette for in situ analysis, comprising an exemplary adapter 300, a biological sample 312 affixed to an unmodified substrate 310, and a cover 306. In FIG. 3E, the cover 306 is placed atop the top surface of the adapter 300 and sandwiched together with the adapter and unmodified substrate. As described above, the hole 308 of the cover 306 is sufficiently larger in area than the hole 302 of the adapter 300 that neither the positional markers and/or fiducial markers 304 of the adapter nor the biological sample positioned within the hole 302 of the adapter is obstructed from view by the body of the cover 306. FIG. 3F further shows a top view of the same fully assembled sample cassette. As illustrated in FIG. 3F, the biological sample 312, the hole 302 of the adapter, and any positional markers and/or fiducial markers 304 on the adapter are fully visible and accessible for any imaging or other in situ technique through the hole 308 of the cover.

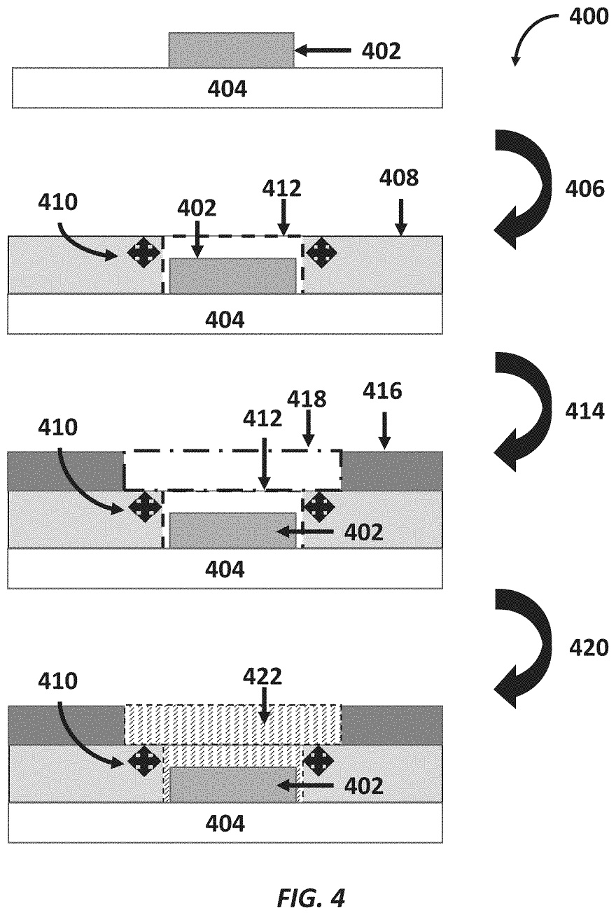

FIG. 4 depicts several cut-out side views of an exemplary process 400 for applying an exemplary adapter to a substrate on which a biological sample has been immobilized, and further assembling an exemplary sample cassette, as shown in FIGS. 3E and 3F. At the beginning of process 400, a biological sample 402 is already affixed to an unmodified substrate 404, which is incompatible with in situ analysis. In step 406, an exemplary adapter 408 comprising positional markers and/or fiducial markers 410 according to one embodiment of the present disclosure is applied to the unmodified substrate 404 and the biological sample 402 affixed thereto. The biological sample 402 sits within a hole 412 of the adapter 408. In step 414, a cover 416 is placed atop the adapter. The cover has a hole 418, which is sufficiently large to allow for visibility and accessibility of the positional markers and/or fiducial markers of the adapter 410, the hole 412 of the adapter, and the biological sample 402. The unmodified substrate 404, the hole of the adapter 412, and the hole of the cover 418, together form a sample well containing the biological sample 402. In step 420, matrix-forming material is delivered to the sample well and polymerized such that the biological sample becomes embedded in a three-dimensional polymerized matrix 422 (e.g., hydrogel matrix). Although the process of FIG. 4 depicts the addition of the cover prior to the delivery of the matrix-forming material and subsequent formation of the three-dimensional matrix, the cover may also be added after the delivery and formation steps.

In one aspect, provided herein is an adapter for a biological sample affixed to substrate for in situ analysis, comprising: a body having a first surface, a second surface, and at least one hole extending through the body from the first surface to the second surface; wherein the first surface comprises one or more positioning markers and/or fiducial markers and wherein the second surface is configured to contact and to be supported by a substrate, wherein the at least one hole is configured in the body to be positioned over the substrate to form at least one sample well, wherein at least one biological sample or a portion thereof is affixed to the substrate and is contained within a sample well.

In another aspect, provided herein is a method of processing a biological sample, comprising a) applying an adapter to a substrate, wherein a biological sample is affixed to the substrate, wherein the adapter comprises a body having a first surface, a second surface, and a first hole extending through the body from the first surface to the second surface; wherein the first surface and/or the second surface comprises one or more positioning markers and/or fiducial markers, wherein the second surface is in contact with the substrate, wherein the first hole is configured to be positioned over the substrate to form a sample well, wherein the biological sample is contained within the sample well; b) applying a cover to the first surface of the adapter, wherein the cover comprises a second body, wherein the second body comprises a third surface, a fourth surface, and a second hole extending through the second body from the third surface to the fourth surface, and wherein the biological sample and the first hole are positioned centrally under the second hole and the second hole has a cross-sectional area such that the biological sample, the first hole, the one or more positioning markers and/or fiducial markers of the first and/or second surface are visible through the second hole; c) delivering a matrix-forming material to the sample well containing the biological sample; and d) forming a three-dimensional polymerized matrix from the matrix-forming material, thereby embedding the biological sample in the three-dimensional polymerized matrix.

In another aspect, provided herein is a sample cassette for in situ analysis, comprising: a biological sample affixed to a substrate; an adapter, comprising a body having a first surface, a second surface, and a first hole extending through the body from the first surface to the second surface, wherein the first surface and/or the second surface comprises one or more positioning markers and/or fiducial markers and wherein the second surface is in contact with and supported by the substrate; wherein the first hole is configured in the body to be positioned over the substrate to form a sample well configured to contain the biological sample affixed to the substrate; and a cover, comprising: a second body having a third surface, a fourth surface, and a second hole extending through the second body from the third surface to the fourth surface, wherein one of the third surface and the fourth surface is in contact with and supported by the second surface of the adapter, and wherein the first hole and second hole are configured such that when the cover is placed atop the first surface of the adapter, the first hole is positioned centrally under the second hole and the second hole has a cross-sectional area such that the first hole and the one or more positioning markers and/or fiducial markers of the first and/or second surface are visible through the second hole.

In yet another aspect, provided herein is a kit, comprising: an adapter, comprising a body having a first surface, a second surface, and a first hole extending through the body from the first surface to the second surface, wherein the first surface and/or second surface comprises one or more positioning markers and/or fiducial markers and wherein the second surface is configured to contact and to be supported by a substrate, wherein the first hole is configured in the body to be positioned over the substrate to form a sample well configured to contain a biological sample affixed to the substrate; a cover, comprising a second body having a third surface, a fourth surface, and a second hole extending through the second body from the third surface to the fourth surface, wherein the first hole and second hole are configured such that when the cover is placed atop the first surface of the adapter, the first hole is positioned centrally under the second hole and the second hole has a cross-sectional area such that the first hole and the one or more positioning markers and/or fiducial markers of the first and/or second surface are visible through the second hole; and optionally, instructions for use thereof.

A. Adapter

In one aspect, provided herein is an adapter for in situ analysis, or more specifically, an adapter for adapting a biological sample affixed to a substrate incompatible with in situ analysis.

In one aspect, provided herein is an adapter for a biological sample affixed to substrate for in situ analysis, comprising: a body having a first surface, a second surface, and at least one hole extending through the body from the first surface to the second surface; wherein the first surface and/or second surface comprises one or more positioning markers and/or fiducial markers and wherein the second surface is configured to contact and to be supported by a substrate, wherein the at least one hole is configured in the body to be positioned over the substrate to form at least one sample well, wherein at least one biological sample or a portion thereof is affixed to the substrate and is contained within a sample well.

In some embodiments, the adapter comprises a planar body and the second surface is configured to contact and be supported by a planar substrate, optionally wherein the planar substrate is a glass slide. In some embodiments, the adapter has a thickness of between about 1 μm and 200 μm. In some embodiments, the body of the adapter has a thickness greater than or equal to the thickness of the at least one biological sample, optionally wherein the at least one biological sample has a thickness between 1 μm and 50 μm or between 5 μm and 20 μm.

In some embodiments, the outer perimeter (or footprint) of the body of the adapter is substantially the same as the outer perimeter (or footprint) of the substrate.

In some embodiments, the first surface of the adapter comprises a coating comprising, or is functionalized with, one or more substances to facilitate attachment of a biological sample (e.g., tissue sample) and/or a three-dimensional polymerized matrix.

In some embodiments, the at least one hole comprises inner walls and wherein the inner walls of the at least one hole comprise a coating comprising, or are functionalized with, one or more substances to facilitate attachment of a biological sample (e.g., tissue sample) and/or a three-dimensional polymerized matrix. In some embodiments, the one or more substances to facilitate attachment comprise lectins, poly-lysine, antibodies, polysaccharides, or binding moieties capable of covalent binding, optionally wherein the one or more substances comprises acryloyls. In some embodiments, the first surface and/or the inner walls of the at least one hole of the adapter comprise a coating comprising, or are functionalized with, one or more substances to deter attachment of one or more analytes, a biological sample (e.g., tissue sample) and/or a three-dimensional polymerized matrix.

In some embodiments, the second surface comprises a coating comprising, or is functionalized with, one or more substances to facilitate attachment to a biological sample (e.g., tissue sample) and/or a three-dimensional polymerized matrix.

In some embodiments, the adapter is made of glass, a rigid polymer (e.g., polystyrene (PS), macroporous polystyrene (MPPS), polymethylmethacrylate (PMMA), polycarbonate (PC), cyclic olefin polymer (COP), or cyclic olefin copolymer (COC)), an elastomer, or adhesive tape, optionally wherein the elastomer is polydimethylsiloxane (PDMS).

B. Adapter Method, Cover and Assembly Steps

(i) Cover

In some embodiments, the present disclosure provides a cover to the adapter as provided herein. In some embodiments, provided herein is a cover wherein the cover comprises a second body, wherein the second body comprises a third surface, a fourth surface, and a second hole extending through the second body from the third surface to the fourth surface, and wherein the biological sample (affixed to the substrate) and the first hole (in the adapter) are positioned centrally under the second hole, and the second hole has a cross-sectional area such that the biological sample, the first hole, and the one or more positioning markers and/or fiducial markers of the first and/or second surface are visible through the second hole.

In some embodiments, the outer perimeter of the cover is substantially the same as the body of the adapter.

In some embodiments, the second hole comprises inner walls. In some embodiments, the inner walls of the second hole comprise a coating comprising, or are functionalized with, one or more substances to facilitate attachment of a three-dimensional polymerized matrix. In some embodiments, the one or more substances to facilitate attachment comprise lectins, poly-lysine, antibodies, polysaccharides, or binding moieties capable of covalent binding, or any combination thereof, optionally wherein the one or more substances comprises acryloyls.

(ii) Assembly Steps

In one aspect, provided herein is a method for processing a biological sample, comprising adapting a biological sample affixed to or immobilized on an unmodified substrate for in situ analysis, wherein the adapting step comprises applying an adapter to the substrate, embedding the sample in a three-dimensional polymerized matrix, and assembling a sample cassette with the substrate, biological sample, adapter and a cover.

In some embodiments, the method comprises applying an adapter to a substrate, wherein a biological sample is affixed to the substrate, wherein the adapter comprises a body having a first surface, a second surface, and a first hole extending through the body from the first surface to the second surface; wherein the first surface and/or second comprises one or more positioning markers and/or fiducial markers, wherein the second surface is in contact with the substrate, and wherein the first hole is configured to be positioned over the substrate to form a sample well, wherein the biological sample is contained within the sample well.

In some embodiments, the substrate to which the biological sample is affixed does not have positioning markers and/or fiducial markers.

In some embodiments, the method comprises delivering a matrix-forming material to the sample well containing the biological sample.

In some embodiments, the method further comprises fixing the biological sample prior to delivering the matrix-forming material to the biological sample immobilized on the substrate. In some embodiments, the method further comprises fixing the biological sample prior to the delivering step in b). In some embodiments, the method further comprises permeabilizing the biological sample prior to delivering the matrix-forming material to the biological sample immobilized on the substrate. In some embodiments, the method further comprises permeabilizing the biological sample prior to the delivering step in b). In other embodiments, the method further comprises permeabilizing the biological sample after fixing the biological sample and prior to delivering the matrix-forming material to the biological sample immobilized on the substrate.

In some embodiments, the step of delivering the matrix-forming material comprises delivering the matrix-forming material to a space between a surface of the substrate to which the biological sample is affixed and a surface of a second substrate, wherein: the space is at least partially enclosed by a spacer, and optionally also the adapter, between the pair of substrates, the space contains the biological sample and the three-dimensional polymerized matrix formed therefrom is sandwiched between the pair of substrates. In some embodiments, the delivering step b) comprises delivering the matrix-forming material to a space between a surface of the substrate to which the biological sample is affixed and a surface of a second substrate, wherein: the space is at least partially enclosed by a spacer between the pair of substrates, the space contains the biological sample, and the three-dimensional polymerized matrix formed in step c) is sandwiched between the pair of substrates. In some embodiments, the spacer is non-integral to the one or both of the pair of substrates. In some embodiments, the spacer is an adhesive tape or ultrathin silicone gasket, optionally wherein the adhesive tape or silicone gasket has a thickness of about 100 μm or between about 10 μm and 20 μm, or comprises a plurality of microparticles, optionally wherein the average diameter of the microparticles is between about 10 μm and 50

In some embodiments, the method comprises forming a three-dimensional polymerized matrix from the matrix-forming material, thereby embedding the biological sample in the three-dimensional polymerized matrix.

In some embodiments, a plurality of molecules in the biological sample are attached (e.g., adsorbed or cross-linked) to the three-dimensional polymerized matrix to substantially retain the relative three-dimensional spatial relationship among the molecules. In some embodiments, the method further comprises forming covalent or non-covalent bonds between the biological sample, or molecules therein, and the three-dimensional polymerized matrix in which the biological sample is embedded.

In some embodiments, the method further comprises applying a cover to the first surface of the adapter, wherein the cover comprises a second body, wherein the second body comprises a third surface, a fourth surface, and a second hole extending through the second body from the third surface to the fourth surface, and wherein the biological sample and the first hole are positioned centrally under the second hole and the second hole has a cross-sectional area such that the biological sample, the first hole, and the one or more positioning markers and/or fiducial markers of the first and/or second surface are visible through the second hole. In some embodiments, the step of applying the cover is performed prior to delivering a matrix-forming material to the sample well containing the biological sample. In some embodiments, the step of applying the cover is performed prior to the delivering step in b). In some embodiments, the step of applying the cover is performed after forming a three-dimensional polymerized matrix from the matrix-forming material, thereby embedding the biological sample in the three-dimensional polymerized matrix. In some embodiments, the step of applying the cover is performed after the forming step in c). In some embodiments wherein a cover is applied, the method further comprises providing a sample cassette for in situ analysis, wherein the sample cassette comprises a biological sample affixed to a substrate and embedded in a three-dimensional polymerized matrix, an adapter, and a cover.

In some embodiments, the method further comprises crosslinking the biological sample embedded in the three-dimensional polymerized matrix. In some embodiments, the method further comprises clearing the biological sample embedded within the three-dimensional polymerized matrix. In some instances, the clearing step may comprise the use of a suitable clearing agent, such as an organic solvent, high refractive index aqueous solution, hyperhydrating solution, detergent, digestion enzyme (e.g., proteinase K) or protein denaturant. In some embodiments, the clearing step comprises contacting the biological sample with a digestion enzyme, optionally wherein the digestion enzyme is proteinase K.

In some embodiments, the method further comprises analyzing one or more analytes (e.g., detecting and/or quantifying the presence of one or more analytes) in the biological sample embedded in the three-dimensional polymerized matrix. In some embodiments, the method further comprises analyzing one or more analytes in the biological sample embedded in the three-dimensional polymerized matrix, wherein the biological sample embedded in the three-dimensional polymerized matrix is contained within a sample cassette.

C. Biological Samples for In Situ Analysis and Sample Cassette

In one aspect, provided herein is a biological sample for in situ analysis obtained according the sample adapter methods of the present disclosure.

In another aspect, provided herein is a sample cassette for in situ analysis, comprising: a biological sample affixed to a substrate; an adapter, comprising a body having a first surface, a second surface, and a first hole extending through the body from the first surface to the second surface, wherein the first surface and/or second surface comprises one or more positioning markers and/or fiducial markers, and wherein the second surface is in contact with and supported by the substrate; wherein the first hole is configured in the body to be positioned over the substrate to form a sample well configured to contain the biological sample affixed to the substrate; and a cover, comprising: a second body having a third surface, a fourth surface, and a second hole extending through the second body from the third surface to the fourth surface, wherein one of the third surface and the fourth surface is in contact with and supported by the second surface of the adapter, and wherein the first hole and second hole are configured such that when the cover is placed atop the first surface of the adapter, the first hole is positioned centrally under the second hole and the second hole has a cross-sectional area such that the first hole and the one or more positioning markers and/or fiducial markers of the first and/or second surface are visible through the second hole.

In still another aspect, provided herein are uses of and methods of using the biological samples for in situ analysis provided herein. In yet another aspect, provided herein are uses of and methods of using the sample cassettes for in situ analysis provided herein. In some embodiments, provided herein are methods of analyzing one or more analytes (e.g., detecting and/or quantifying the presence of one or more analytes) in the biological sample of the sample cassette provided herein.

### IV. Spacers

As provided in the present disclosure, the methods for processing a biological sample for in situ analysis may comprise steps for the formation of one or more three-dimensional polymerized matrices. In some embodiments of the preceding aspects, the three-dimensional polymerized matrices having thicknesses less than about 200 μm may be achieved by employing readily available spacer materials to control the thickness of the three-dimensional polymerized matrices as they are formed. In some embodiments, the spacer is an adhesive tape, ultrathin silicone gasket, or a plurality of microparticles.

A. Adhesive Tape

In some embodiments, the spacer is an adhesive tape. The adhesive tape may be characterized by its chemical composition and dimensions, such as thickness. The adhesive tape to be used as a spacer as provided in the methods herein is preferably non-reactive and/or inert to any biological samples, matrix-forming materials or associated reagents for sample processing or polymerization.

In some embodiments, the adhesive tape has a thickness of about 200 μm, about 150 μm, about 100 μm, about 50 μm, about 20 μm, about 10 μm, or about 5 μm. In other embodiments, the adhesive tape has a thickness of less than or equal to about 200 μm, less than or equal to about 150 μm, less than or equal to about 100 μm, less than or equal to about 50 μm, less than or equal to about 20 μm, or less than or equal to about 10 μm. In yet other embodiments, the adhesive tape has a thickness of greater than or equal to about 100 μm, greater than or equal to about 50 μm, greater than or equal to about 20 μm, greater than or equal to about 10 μm, or greater than or equal to about 5 μm.

In some embodiments, the adhesive tape has a thickness between about 5 μm and about 200 μm, between about 5 μm and about 150 μm, between about 5 μm and about 100 μm, between about 5 μm and about 50 μm, between about 5 μm and about 20 μm, between about 5 μm and about 10 μm, between about 10 μm and about 200 μm, between about 10 μm and about 150 μm, between about 10 μm and about 100 μm, between about 10 μm and about 50 μm, between about 10 μm and about 20 μm, between about 20 μm and about 200 μm, between about 20 μm and about 150 μm, between about 20 μm and about 100 μm, or between about 20 μm and about 50 μm. In other embodiments, the adhesive tape has a thickness between about 5 μm and about 200 μm. In other embodiments, the adhesive tape has a thickness between about 10 μm and about 100 μm, between about 10 μm and about 20 μm or between about 5 μm and about 20 μm.

In some embodiments, the spacer is an adhesive tape, optionally wherein the adhesive tape has a thickness of about 100 μm or between about 10 μm and 20 μm.

B. Ultrathin Silicone Gaskets

In some embodiments, the spacer is an ultrathin silicone gasket. The silicone gasket may be characterized by its chemical composition and dimensions, such as thickness. The silicone gasket to be used as a spacer as provided in the methods herein is preferably non-reactive and/or inert to any biological samples, matrix-forming materials or associated reagents for sample processing or polymerization. In some embodiments, ultrathin gaskets may be cut from silicone rubber sheet materials (such as those available from TYM Seals & Gaskets, Ltd., Devizes, UK) or from other ultrathin sheet materials.

In some embodiments, the silicone gasket has a thickness of about 200 μm, about 150 μm, about 100 μm, about 50 μm, about 20 μm, about 10 μm, or about 5 μm. In other embodiments, the silicone gasket has a thickness of less than or equal to about 200 μm, less than or equal to about 150 μm, less than or equal to about 100 μm, less than or equal to about 50 μm, less than or equal to about 20 μm, or less than or equal to about 10 μm. In yet other embodiments, the silicone gasket has a thickness of greater than or equal to about 100 μm, greater than or equal to about 50 μm, greater than or equal to about 20 μm, greater than or equal to about 10 μm, or greater than or equal to about 5 μm.

C. Plurality of Microparticles

In some embodiments, the spacer comprises a plurality of microparticles, such as sufficiently rigid microparticles, that may be positioned between a first and a second substrate to control spacing. The plurality of microparticles to be used as a spacer as provided in the methods herein are preferably non-reactive and/or inert to any biological samples, matrix-forming materials or associated reagents for sample processing or polymerization.

The plurality of microparticles may be characterized by their chemical composition, shape and/or dimensions, and other size distribution properties, such as average diameter or poly- or monodispersity. The plurality of microparticles to be used as a spacer as provided in the methods herein are preferably non-reactive and/or inert to any biological samples, matrix-forming materials or associated reagents for sample processing or polymerization.

In some embodiments, the plurality of microparticles comprises a plurality of rigid silica microparticles. In some embodiments, the plurality of microparticles is monodisperse. In other embodiments, the plurality of microparticles is polydisperse. In other embodiments, the plurality of microparticles comprises a plurality of silica monospheres.

In certain embodiments, the plurality of microparticles may be characterized by their size distribution, an average value for a given dimension, or a range of values for a given dimension. For example, in some embodiments, the plurality of microparticles may be characterized by an average diameter.

In some embodiments, the plurality of microparticles has an average diameter of about 200 μm, about 150 μm, about 100 μm, about 50 μm, about 20 μm, about 10 μm, or about 5 μm. In other embodiments, the plurality of microparticles has an average diameter of less than or equal to about 200 μm, less than or equal to about 150 μm, less than or equal to about 100 μm, less than or equal to about 50 μm, less than or equal to about 20 μm, or less than or equal to about 10 μm. In yet other embodiments, the plurality of microparticles has an average diameter of greater than or equal to about 100 μm, greater than or equal to about 50 μm, greater than or equal to about 20 μm, greater than or equal to about 10 μm, or greater than or equal to about 5

In some embodiments, the plurality of microparticles has an average diameter between about 5 μm and about 200 μm, between about 5 μm and about 150 μm, between about 5 μm and about 100 μm, between about 5 μm and about 50 μm, between about 5 μm and about 20 μm, between about 5 μm and about 10 μm, between about 10 μm and about 200 μm, between about 10 μm and about 150 μm, between about 10 μm and about 100 μm, between about 10 μm and about 50 μm, between about 10 μm and about 20 μm, between about 20 μm and about 200 μm, between about 20 μm and about 150 μm, between about 20 μm and about 100 μm, or between about 20 μm and about 50 μm. In other embodiments, the plurality of microparticles has an average diameter between about 5 μm and about 200 μm. In other embodiments, the plurality of microparticles has an average diameter between about 10 μm and about 100 μm, between about 10 μm and about 20 μm or between about 5 μm and about 20 μm.

In some embodiments, the spacer comprises a plurality of microparticles, optionally wherein the average diameter of the microparticles is between about 10 μm and 50 μm.

In some embodiments, the plurality of microparticles are uniform in size. In some embodiments, the diameters of at least about 80%, at least about 85%, at least about 90%, at least about 95%, or at least about 99% of the microparticles are the average diameter of the plurality of microparticles. In some embodiments, the standard deviation of the diameters of the plurality of microparticles is no more than 5 μm, no more than 4 μm, no more than 3 μm, no more than 2 μm, no more than 1 μm, no more than 0.5 μm, or no more than 0.1 μm.

D. Application of Spacers, Formation of Space Between Substrates, and/or Removal of Spacers

For the methods of the present disclosure, the spacers are easily applied to one substrate of a pair of substrates, and optionally also easily removed from the same, which obviates the need for expensive engineering controls to pre-fabricate casting molds having precise dimensions and pre-formed spacers. As detailed above, the spacer sets the distance (or space) between the two substrates once they are sandwiched together, which in turn sets the maximum thickness of any three-dimensional polymerized matrix that can form in the space between the two substrates. In some embodiments, the spacer is removably affixed to one of the first substrate and the second substrate. In some embodiments, the spacer is integral to one of the first or second substrates. In some embodiments, the spacer is non-integral to both the first substrate and the second substrate.

In some embodiments, the methods provided herein further comprise steps for forming the space between the first and second substrates, including but not limited to applying a spacer to one of the first and second substrates and bringing together the first and second substrates with the spacer sandwiched between the surfaces of the first and second substrates, such that a space is formed between them.

In some embodiments, the methods provided herein comprise applying the spacer to the surface of the first substrate and/or the surface of the second substrate, and bringing the first substrate and second substrate together such that the spacer separates the first and second substrates to provide the space.

In some embodiments wherein the spacer is an adhesive tape or ultrathin silicone gasket, the applying step comprises affixing the adhesive tape or silicone gasket to the surface of either the first substrate or second substrate.

In some embodiments wherein the spacer comprises a plurality of microparticles, the applying step comprises adding a suspension to the surface of either the first substrate or second substrate, wherein the suspension comprises the plurality of microparticles and a carrier. In some embodiments, the carrier comprises mineral oil.

In some embodiments, the plurality of microparticles are not mixed with the matrix-forming material and do not contact the three-dimensional polymerized matrix. In some embodiments wherein the spacer comprises a plurality of microparticles, the step of delivering the matrix-forming material to the space between the surface of the first substrate and the surface of a second substrate comprises adding the matrix-forming material to the surface of the first substrate or the surface of a second substrate, adding a suspension of microparticles to the surface of the first substrate or the surface of the second substrate to which the matrix-forming material was added, and bringing the first substrate and second substrate together such that the spacer separates the first and second substrates to provide the space.

In some embodiments wherein the applying step comprises adding a suspension to the surface of either the first substrate or second substrate, wherein the suspension comprises the plurality of microparticles and a carrier, the method further comprises evaporating the carrier to provide the plurality of microparticles deposited on the surface of the first substrate or second substrate.

The space produced by the sandwiching of the first and second substrates, and into which the matrix-forming material is delivered, is largely characterized by the dimension along the z-axis of the space (e.g., the axis that is normal to the planar surfaces of the first and second substrates), which is the distance between the surfaces of the first and second substrates. In some embodiments, the space may be further characterized by its dimensions parallel to the surface of the two substrates, such as the dimensions in the x- and y-axes, and/or the total volume of space formed by the two substrates and the spacer, such as in cubic millimeters.

In some embodiments, the distance between the surfaces of the first and second substrates is substantially equal to the thickness of the adhesive tape or silicone gasket, or substantially equal to the average diameter of the microparticles. In some embodiments, the distance between the surfaces of the first and second substrates is substantially equal to the thickness of the adhesive tape or silicone gasket. In some embodiments, the distance between the surfaces of the first and second substrates is substantially equal to the average diameter of the microparticles.

In some embodiments, the three-dimensional polymerized matrix comprises a biological sample or a biological sample is embedded in the three-dimensional polymerized matrix. In some embodiments wherein the three-dimensional polymerized matrix comprises a biological sample or a biological sample is embedded in the three-dimensional polymerized matrix, the space between the first substrate and the second substrate may comprise a biological sample. In some embodiments, the space between the first substrate and the second substrate comprises a biological sample (e.g., tissue sample), wherein the biological sample (e.g., tissue sample) is immobilized or affixed to the surface of one of the first and second substrates.

In some embodiments, the biological sample is immobilized or affixed to the surface of one of the first and second substrates prior to the step of delivering the matrix-forming material to the space. In other embodiments, the biological sample is immobilized or affixed to the surface of one of the first and second substrates prior to the step of applying the spacer material to the surface of one of the first and second substrates.

As described above, in some instances the spacer is non-integral to either the first or second substrate and is removably affixed. Following the formation of the three-dimensional polymerized matrix from the matrix-forming material, the spacer, the first substrate and the second substrate may be disassembled from their sandwich-type configuration to provide the three-dimensional polymerized matrix, or biological sample embedded in the three-dimensional polymerized matrix, for further downstream applications, such as characterization or analysis.

In some embodiments, the methods provided herein comprise removing the spacer from the first and/or the second substrates. In some embodiments, the spacer is applied to the second substrate which is downward facing, and the method further comprises removing the second substrate from the first substrate, thereby removing the spacer at least partially enclosing the biological sample. In other embodiments, the spacer is applied to the first substrate which is upward facing, and the method further comprises removing the second substrate from the first substrate, and optionally further removing the spacer from the first substrate.

### V. Three-Dimensional Polymerized Matrices, Matrix-Forming Materials, and Polymerization

As provided in the present disclosure, the methods described herein may be used to prepare extremely thin three-dimensional polymerized matrices, particularly for the preparation of biological samples embedded within said three-dimensional polymerized matrices for subsequent in situ analysis.

A. Three-Dimensional Polymerized Matrices and Matrix-Forming Materials

In some embodiments of the present disclosure, the methods provided herein are methods for preparing a three-dimensional polymerized matrix.

As described above, the three-dimensional matrices obtainable by the methods of the present disclosure can be produced with thicknesses less than those obtained by existing methods.

In some embodiments, the three-dimensional polymerized matrix has an average thickness of about 200 μm, about 150 μm, about 100 μm, about 50 μm, about 20 μm, about 10 μm, or about 5 μm. In other embodiments, the three-dimensional polymerized matrix has an average thickness of less than or equal to about 200 μm, less than or equal to about 150 μm, less than or equal to about 100 μm, less than or equal to about 50 μm, less than or equal to about 20 μm, or less than or equal to about 10 μm. In yet other embodiments, the three-dimensional polymerized matrix has an average thickness of greater than or equal to about 100 μm, greater than or equal to about 50 μm, greater than or equal to about 20 μm, greater than or equal to about 10 μm, or greater than or equal to about 5 μm.

In some embodiments, the three-dimensional polymerized matrix has an average thickness between about 5 μm and about 200 μm, between about 5 μm and about 150 μm, between about 5 μm and about 100 μm, between about 5 μm and about 50 μm, between about 5 μm and about 20 μm, between about 5 μm and about 10 μm, between about 10 μm and about 200 μm, between about 10 μm and about 150 μm, between about 10 μm and about 100 μm, between about 10 μm and about 50 μm, between about 10 μm and about 20 μm, between about 20 μm and about 200 μm, between about 20 μm and about 150 μm, between about 20 μm and about 100 μm, or between about 20 μm and about 50 μm. In other embodiments, the three-dimensional polymerized matrix has an average thickness between about 5 μm and about 200 μm. In other embodiments, the three-dimensional polymerized matrix has an average thickness between about 10 μm and about 100 μm, between about 10 μm and about 20 μm or between about 5 μm and about 20 μm.

In some aspects, the thickness of the three-dimensional polymerized matrix generated using a method disclosed herein is no more than about 200 μm, no more than about 150 μm, or no more than about 100 μm. For instance, the thickness of the polymer matrix can be between about 15 μm and about 100 μm. In some aspects, the difference between the thickness of the polymer matrix and the thickness of a tissue sample embedded in the polymer matrix is no more than about 150 μm, no more than about 125 μm, no more than about 100 μm, no more than about 90 μm, no more than about 80 μm, no more than about 70 μm, no more than about 60 μm, no more than about 50 μm, no more than about 40 μm, no more than about 30 μm, no more than about 25 μm, no more than about 20 μm, no more than about 15 μm, no more than about 10 μm, no more than about 5 μm, or no more than about 2 μm. In some aspects, the thickness of the polymer matrix is no more than 15 times, no more than 14 times, no more than 13 times, no more than 12 times, no more than 11 times, no more than 10 times, no more than 9 times, no more than 8 times, no more than 7 times, no more than 6 times, no more than 5 times, no more than 4 times, no more than 3 times, no more than 2 times, no more than 1.5 times, no more than 1.2 times, or no more than 1.1 times of the thickness of a tissue sample embedded in the polymer matrix.

In some embodiments, a three-dimensional polymerized matrix may be formed with the assistance of a spacer to control the thickness of the three-dimensional polymerized matrix. The spacer sets the distance (or space) between a pair of substrates (such as a first substrate and a second substrate, or a second substrate and a third substrate) once they are sandwiched together, which in turn sets the maximum thickness of any three-dimensional polymerized matrix that can form in the space between the pair of substrates. By virtue of the spacer material, the average thickness of the three-dimensional polymerized matrix can be controlled such that the average thickness of the three-dimensional polymerized matrix is less than or equal to the distance between the pair of substrates, and by proxy, when adhesive tape is the spacer, less than or equal to the average thickness of adhesive tape, or, when microparticles are the spacer, less than or equal to the average diameter of the microparticles.

The three-dimensional polymerized matrix is formed from a matrix-forming material, under suitable conditions, such as polymerization conditions. Depending upon the nature and physical and chemical properties of the three-dimensional polymerized matrix desired, the matrix-forming materials may be selected accordingly.

In some embodiments, the three-dimensional polymerized matrix is a hydrogel matrix. In some embodiments, the three-dimensional polymerized matrix comprises a biological sample embedded therein. In certain embodiments, the three-dimensional polymerized matrix is a hydrogel matrix comprising a biological sample embedded therein.

In some embodiments, the matrix-forming material comprises monomer units which are capable polymerizing to form a three-dimensional polymerized matrix. In certain embodiments, the matrix-forming material comprises hydrogel monomers, or subunits. In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof. In some embodiments, the matrix-forming material comprises polyacrylamide, cellulose, alginate, polyamide, cross-linked agarose, cross-linked dextran or cross-linked polyethylene glycol. In some embodiments, the first matrix-forming material and/or second matrix-forming material comprises polyacrylamide, cellulose, alginate, polyamide, cross-linked agarose, cross-linked dextran or cross-linked polyethylene glycol. In some embodiments, the first matrix-forming material and second matrix-forming material may be the same or different.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

B. Additional Components

In some embodiments, the three-dimensional polymerized matrices of the present disclosure may comprise one or more additional components suitable for polymerization, downstream processing and/or characterization.

In some embodiments, the matrix-forming material comprises a plurality of stimulus-responsive matrix-forming monomers and wherein the first three-dimensional polymerized matrix expands when exposed to a suitable stimulus, optionally wherein the stimulus-responsive matrix-forming monomers comprise a sodium acrylate monomer and/or an N-isopropylacrylamide/acrylamide monomer. In some embodiments, the first matrix-forming material comprises a plurality of stimulus-responsive matrix-forming monomers and wherein the first three-dimensional polymerized matrix expands when exposed to a suitable stimulus, optionally wherein the stimulus-responsive matrix-forming monomers comprise a sodium acrylate monomer and/or an N-isopropylacrylamide/acrylamide monomer. In some embodiments, the plurality of stimulus-responsive matrix-forming monomers comprises acrylic acid and acrylamide monomers, or N-isopropylacrylamide and acrylamide monomers.

In some embodiments, the first matrix-forming material does not comprise a plurality of stimulus-responsive matrix-forming monomers. In some embodiments, the second matrix-forming material does not comprise a plurality of stimulus-responsive matrix-forming monomers. In some embodiments, the first matrix-forming material comprises acrylamide and N,N-methylenebisacrylamide (BIS), or acrylamide, polydopamine (PDA) and N,N′-diallyltartardiamide (DATD).

In some embodiments, the first matrix-forming material comprises a plurality of matrix-forming monomers comprising functional groups capable of forming covalent bonds with one or more molecules in the biological sample.

In some embodiments, the second matrix-forming material comprises one or more cross-linking agents or a plurality of matrix-forming monomers capable of forming cross-linking bonds. In some embodiments, the one or more cross-linking agents comprises N,N,N′,N′-tetramethylethylenediamine (TEMED), N,N-methylenebisacrylamide (BIS), or N,N′-diallyltartardiamide (DATD). In other embodiments, the plurality of matrix-forming monomers capable of forming cross-linking bonds comprises matrix-forming monomers comprising Click-compatible moieties, optionally wherein the Click-compatible moieties are azido and/or alkynyl moieties.

In some embodiments, the second matrix-forming material further comprises one or more initiators, optionally wherein the one or more initiators comprises potassium peroxodisulfate or ammonium sulfate.

In some embodiments, the matrix-forming material comprises a plurality of fluorescent beads. In certain embodiments, the matrix-forming material comprises a plurality of fluorescent beads, optionally wherein the fluorescent beads have an average diameter of about 0.2 μm. In certain embodiments, the first and/or second matrix-forming material comprises a plurality of fluorescent beads, optionally wherein the fluorescent beads have an average diameter of about 0.2 μm. In some embodiments, the matrix-forming material comprises a plurality of fluorescent beads, and wherein the thickness of the hydrogel matrix is measured by imaging the fluorescent beads, optionally wherein the fluorescent beads have an average diameter of about 0.2 μm. In some embodiments, the first and/or second matrix-forming material comprises a plurality of fluorescent beads, and wherein the thickness of the hydrogel matrix is measured by imaging the fluorescent beads, optionally wherein the fluorescent beads have an average diameter of about 0.2 μm.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

C. Polymerization

The present disclosure provides for methods of processing biological samples for in situ analysis. In one aspect, provided herein are methods for transferring a biological sample from a first substrate to a second substrate by embedding the biological sample in a first three-dimensional polymerized matrix, further embedding the embedded sample in a second three-dimensional polymerized matrix and immobilizing the second three-dimensional polymerized matrix to the second substrate. In some embodiments, the first three-dimensional polymerized matrix is formed from a first matrix-forming material and the second three-dimensional polymerized matrix is formed from a second matrix-forming material. In some embodiments, the first three-dimensional polymerized matrix and the second three-dimensional polymerized matrix are formed by subjecting the first matrix-forming material and second matrix-forming material to polymerization.

In another aspect, provided herein are methods for adapting a biological sample for in situ analysis by applying an adapter to the substrate on which the biological sample is immobilized, and further embedding the sample in a three-dimensional polymerized matrix. In some embodiments, the three-dimensional polymerized matrix is formed from a matrix-forming material. In some embodiments, the three-dimensional polymerized matrix is formed by subjecting the matrix-forming material and second matrix-forming material to polymerization.

Depending upon the matrix-forming material employed, the conditions for polymerization may vary accordingly across a number of variables including but not limited to polymerization reagents, reaction temperature, and reaction time. Suitable polymerization conditions for any of the matrix-forming materials as provided herein may be used for the methods of the present disclosure.

In some embodiments, the polymerization is initiated by adding a polymerization-inducing catalyst, UV light or functional cross-linkers. In some embodiments, polymerization is initiated by adding a polymerization-inducing catalyst. In some embodiments, polymerization is initiated by adding UV light. In certain embodiments, the UV light has a wavelength between 200 nm and 400 nm. In some embodiments, polymerization is initiated by adding one or more functional cross-linkers.

Although the thickness of the three-dimensional matrices is largely controlled by the dimensions of the spacer materials, constant pressure or force may be applied to the sandwiched substrates containing the matrix-forming material before and during the formation of the matrix in order to achieve the desired thickness of the resulting matrix. For example, the application of a force to the first and/or second (or third, if applicable) substrates may facilitate the expulsion of excess matrix-forming material from the space between the two substrates, prevent any volume expansion by the matrix-forming material during the matrix formation, and maintain the desired distance between the two substrates to control the final thickness In some embodiments, the method comprises applying a force to the first and/or second substrates prior to forming the three-dimensional polymerized matrix.

### VI. Samples and Sample Processing

A. Embedding of Biological Samples

In some embodiments, the three-dimensional polymerized matrix comprises a biological sample. In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments of the methods for transferring a sample provided herein, a biological sample is embedded in a first three-dimensional polymerized matrix, which is further embedded in a second three-dimensional polymerized matrix.

In some embodiments of the methods for adapting a sample provided herein, a biological sample is embedded in a three-dimensional polymerized matrix.

In some embodiments, the biological sample is immobilized on the surface of the first or unmodified substrate. In some embodiments, the method comprises fixing the biological sample prior to the delivering step. In other embodiments, the method comprises permeabilizing the biological sample after the fixing step and prior to the delivering step. In some embodiments, the method comprises cross-linking the biological sample embedded within the three-dimensional polymerized matrix. In some embodiments of the methods provided herein, the method comprises clearing the biological sample embedded within the three-dimensional polymerized matrix.

In some embodiments, the method comprises applying a spacer to a surface of a first substrate or a surface of a second substrate, wherein the spacer is non-integral to the first or second substrate, and wherein a biological sample is immobilized on the surface of a first substrate. In some embodiments, the method further comprises bringing the first and second substrates together to form a space between the first and second substrates such that the biological sample is in the space at least partially enclosed by the spacer. In some embodiments, the method comprises delivering a matrix-forming material in the space. In still further embodiments, the method comprises forming a three-dimensional polymerized matrix from the matrix-forming material in the space, thereby embedding the biological sample in the three-dimensional polymerized matrix. In some embodiments, the three-dimensional polymerized matrix has an average thickness between about 10 μm and about 100 μm.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen, et al., Science 347(6221):543-548, 2015, the entire contents of which are incorporated herein by reference.

B. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a predisposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., about 55, about 60, about 65, about 70, about 75, about 80, about 85, about 90, about 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analyzed. In some embodiments, the biological sample is a tissue slice between about 1 μm and about 50 μm in thickness, optionally wherein the tissue slice is between about 5 μm and about 35 μm in thickness. In some embodiments, the biological sample is a tissue slice about 5 μm, about 10 μm, about 15 μm, about 20 μm, about 25 μm, about 30 μm, about 35 μm, about 40 μm, about 45 μm, or about 50 μm in thickness.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

Biological samples can also include fetal cells. For example, a procedure such as amniocentesis can be performed to obtain a fetal cell sample from maternal circulation. Sequencing of fetal cells can be used to identify any of a number of genetic disorders, including, e.g., aneuploidy such as Down's syndrome, Edwards syndrome, and Patau syndrome. Further, cell surface features of fetal cells can be used to identify any of a number of disorders or diseases.

Biological samples can also include immune cells. Sequence analysis of the immune repertoire of such cells, including genomic, proteomic, and cell surface features, can provide a wealth of information to facilitate an understanding the status and function of the immune system. By way of example, determining the status (e.g., negative or positive) of minimal residue disease (MRD) in a multiple myeloma (MM) patient following autologous stem cell transplantation is considered a predictor of MRD in the MM patient (see, e.g., U.S. Patent Application Publication No. 2018/0156784, the entire contents of which are incorporated herein by reference).

Examples of immune cells in a biological sample include, but are not limited to, B cells, T cells (e.g., cytotoxic T cells, natural killer T cells, regulatory T cells, and T helper cells), natural killer cells, cytokine induced killer (CIK) cells, myeloid cells, such as granulocytes (basophil granulocytes, eosinophil granulocytes, neutrophil granulocytes/hypersegmented neutrophils), monocytes/macrophages, mast cells, thrombocytes/megakaryocytes, and dendritic cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

C. Pre-Embedding and Post-Embedding Steps

In some embodiments, the methods provided herein for processing a biological sample may further comprise additional steps to obtain and/or prepare the biological samples prior to forming a three-dimensional matrix or embedding the biological sample in any three-dimensional matrix. It should be recognized that the biological samples as provided herein may be subjected to various processing steps, the combinations of which are used to obtain and applied to each biological sample may vary depending upon the type of biological sample, the source of the biological sample and the intended analytical method for the biological sample, among others. Relevant processing steps may include but are not limited to tissue sectioning, freezing, fixation and post-fixation, permeabilizing, staining, expansion, cross-linking, de-cross-linking, and/or clearing.

In some embodiments, the methods provided herein for preparing a three-dimensional matrix and/or embedding a biological sample in a three-dimensional matrix may further comprise additional steps to prepare the biological samples after to forming the three-dimensional matrix or embedding the biological sample in the three-dimensional matrix. As with pre-processing steps applied to the biological samples, it should be recognized that the biological samples as provided herein may be subjected to various processing steps, the combinations of which are used to applied to each biological sample embedded in the three-dimensional polymerized matrix may vary depending upon the type of biological sample, the source of the biological sample and the intended analytical method for the biological sample, among others. Relevant processing steps may include but are not limited to fixation and post-fixation, permeabilizing, staining, expansion, cross-linking, de-cross-linking, and/or clearing.

In some embodiments, one or more processing steps disclosed in this section, e.g., in sections V-C-(i) to V-C-(ix), can be performed prior to, during, and/or after the embedding step.

(i) Tissue Sectioning

In some embodiments, the method further comprises obtaining a biological sample to be embedded in the three-dimensional polymerized matrix.

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analyzed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

(ii) Freezing

In some embodiments, the biological sample (e.g., a tissue section as described above) can be prepared by deep freezing at a temperature suitable to maintain or preserve the integrity (e.g., the physical characteristics) of the tissue structure. Such a temperature can be, e.g., less than −20° C., or less than −25° C., −30° C., −40° C., −50° C., −60° C., −70° C., −80° C. −90° C., −100° C., −110° C., −120° C., −130° C., −140° C., −150° C., −160° C., −170° C., −180° C., −190° C., or −200° C. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Post-Fixation

In some embodiments, the method comprises fixing the biological sample prior to delivering matrix-forming material.

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as post-fixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

(iv) Disaggregation of Cells

In some embodiments, the biological sample corresponds to cells (e.g., derived from a cell culture, a tissue sample, or cells deposited on a surface). In a cell sample with a plurality of cells, individual cells can be naturally unaggregated. For example, the cells can be derived from a suspension of cells and/or disassociated or disaggregated cells from a tissue or tissue section.

Alternatively, the cells in the sample may be aggregated, and may be disaggregated into individual cells using, for example, enzymatic or mechanical techniques. Examples of enzymes used in enzymatic disaggregation include, but are not limited to, dispase, collagenase, trypsin, and combinations thereof. Mechanical disaggregation can be performed, for example, using a tissue homogenizer. The biological sample may comprise disaggregated cells (e.g., nonadherent or suspended cells) which are deposited on a surface and subjected to an in situ assay and a spatial assay disclosed herein.

(v) Tissue Permeabilization and Treatment

In some embodiments, the method comprises permeabilizing the biological sample prior to the delivering of matrix-forming material to the biological sample immobilized on a first or unmodified substrate. In some embodiments, the method comprises permeabilizing the biological sample after the fixing step and prior to the delivering of the matrix-forming material to the biological sample immobilized on a first or unmodified substrate.

In some embodiments, a biological sample can be permeabilized to facilitate transfer of analytes out of the sample, and/or to facilitate transfer of species (such as probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, where a diffusion-resistant medium is used to limit migration of analytes or other species during the analytical procedure, the diffusion-resistant medium can include at least one permeabilization reagent. For example, the diffusion-resistant medium can include wells (e.g., micro-, nano-, or picowells) containing a permeabilization buffer or reagents. In some embodiments, where the diffusion-resistant medium is a hydrogel, the hydrogel can include a permeabilization buffer. In some embodiments, the hydrogel is soaked in permeabilization buffer prior to contacting the hydrogel with a sample. In some embodiments, the hydrogel or other diffusion-resistant medium can contain dried reagents or monomers to deliver permeabilization reagents when the diffusion-resistant medium is applied to a biological sample. In some embodiments, the diffusion-resistant medium, (i.e. hydrogel) is covalently attached to a solid substrate (i.e. an acrylated glass slide). In some embodiments, the hydrogel can be modified to both contain capture probes and deliver permeabilization reagents. For example, a hydrogel film can be modified to include spatially-barcoded capture probes. The spatially-barcoded hydrogel film is then soaked in permeabilization buffer before contacting the spatially-barcoded hydrogel film to the sample. The spatially-barcoded hydrogel film thus delivers permeabilization reagents to a sample surface in contact with the spatially-barcoded hydrogel, enhancing analyte migration and capture. In some embodiments, the spatially-barcoded hydrogel is applied to a sample and placed in a permeabilization bulk solution. In some embodiments, the hydrogel film soaked in permeabilization reagents is sandwiched between a sample and a spatially-barcoded array. In some embodiments, target analytes are able to diffuse through the permeabilizing reagent soaked hydrogel and hybridize or bind the capture probes on the other side of the hydrogel. In some embodiments, the thickness of the hydrogel is proportional to the resolution loss. In some embodiments, wells (e.g., micro-, nano-, or picowells) can contain spatially-barcoded capture probes and permeabilization reagents and/or buffer. In some embodiments, spatially-barcoded capture probes and permeabilization reagents are held between spacers. In some embodiments, the sample is punch, cut, or transferred into the well, wherein a target analyte diffuses through the permeabilization reagent/buffer and to the spatially-barcoded capture probes. In some embodiments, resolution loss may be proportional to gap thickness (e.g. the amount of permeabilization buffer between the sample and the capture probes). In some embodiments, the diffusion-resistant medium (e.g. hydrogel) is between approximately 50-500 micrometers thick including 500, 450, 400, 350, 300, 250, 200, 150, 100, or 50 micrometers thick, or any thickness within 50 and 500 micrometers.

In some embodiments, permeabilization solution can be delivered to a sample through a porous membrane. In some embodiments, a porous membrane is used to limit diffusive analyte losses, while allowing permeabilization reagents to reach a sample. Membrane chemistry and pore size can be manipulated to minimize analyte loss. In some embodiments, the porous membrane may be made of glass, silicon, paper, hydrogel, polymer monoliths, or other material. In some embodiments, the material may be naturally porous. In some embodiments, the material may have pores or wells etched into solid material. In some embodiments, the permeabilization reagents are flowed through a microfluidic chamber or channel over the porous membrane. In some embodiments, the flow controls the sample's access to the permeabilization reagents. In some embodiments, a porous membrane is sandwiched between a spatially-barcoded array and the sample, wherein permeabilization solution is applied over the porous membrane. The permeabilization reagents diffuse through the pores of the membrane and into the tissue.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

(vi) Staining

To facilitate visualization, biological samples can be stained using a wide variety of stains and staining techniques. In some embodiments, for example, a sample can be stained using any number of stains, including but not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, propidium iodide, rhodamine, or safranine.

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of destaining or discoloring a biological sample generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

(vii) Isometric Expansion

In some embodiments, the matrix-forming material comprises a plurality of stimulus-responsive matrix-forming monomers and wherein the first three-dimensional polymerized matrix expands when exposed to a suitable stimulus, optionally wherein the stimulus-responsive matrix-forming monomers comprise a sodium acrylate monomer and/or an N-isopropylacrylamide/acrylamide monomer. In some embodiments, the plurality of stimulus-responsive matrix-forming monomers comprises acrylic acid and acrylamide monomers, or N-isopropylacrylamide and acrylamide monomers. In some embodiments, the methods provided herein comprise expanding the biological sample embedded in the (first) three-dimensional polymerized matrix. In some embodiments, the expanding step comprises subjecting the first three-dimensional polymerized matrix to a change in salt concentration or a temperature change.

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(viii) Crosslinking and De-Crosslinking

In some embodiments, the methods provided herein comprise crosslinking the biological sample embedded in the three-dimensional polymerized matrix. In some embodiments, a plurality of molecules in the biological sample are attached to the three-dimensional polymerized matrix to substantially retain the relative three-dimensional spatial relationship among the molecules.

In some embodiments, the biological sample is reversibly cross-linked prior to or during an in situ assay round. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method. A hydrogel may include a macromolecular polymer gel including a network. Within the network, some polymer chains can optionally be cross-linked, although cross-linking does not always occur.

Where the substrate includes a gel (e.g., a hydrogel or gel matrix), oligonucleotides within the gel can attach to the substrate. The terms “hydrogel” and “hydrogel matrix” are used interchangeably herein to refer to a macromolecular polymer gel including a network. Within the network, some polymer chains can optionally be cross-linked, although cross-linking does not always occur.

In some embodiments, the substrate has a conditionally removable coating (e.g., a coating that can be removed from the surface of a substrate upon application of a releasing agent). In some embodiments, a conditionally removable coating includes a hydrogel as described herein, e.g., a hydrogel including a polypeptide-based material. Non-limiting examples of a hydrogel featuring a polypeptide-based material include a synthetic peptide-based material featuring a combination of spider silk and a trans-membrane segment of human muscle L-type calcium channel (e.g., PEPGEL®), an amphiphilic 16 residue peptide containing a repeating arginine-alanine-aspartate-alanine sequence (RADARADARADARADA, SEQ ID NO: 1) (e.g., PURAMATRIX®), EAK16 (AEAEAKAKAEAEAKAK, SEQ ID NO: 2), KLD12 (KLDLKLDLKLDL, SEQ ID NO: 3), and PGMATRIX™.

In some embodiments, the hydrogel in the conditionally removable coating is a stimulus-responsive hydrogel. A stimulus-responsive hydrogel can undergo a gel-to-solution and/or gel-to-solid transition upon application of one or more external triggers (e.g., a releasing agent). See, e.g., Willner, Acc. Chem. Res. 50:657-658, 2017, which is incorporated herein by reference in its entirety. Non-limiting examples of a stimulus-responsive hydrogel include a thermoresponsive hydrogel, a pH-responsive hydrogel, a light-responsive hydrogel, a redox-responsive hydrogel, an analyte-responsive hydrogel, or a combination thereof. In some embodiments, a stimulus-responsive hydrogel can be a multi-stimuli-responsive hydrogel.

In some embodiments, a “releasing agent” or “external trigger” is an agent that allows for the removal of a conditionally removable coating from a substrate when the releasing agent is applied to the conditionally removable coating. An external trigger or releasing agent can include physical triggers such as thermal, magnetic, ultrasonic, electrochemical, and/or light stimuli as well as chemical triggers such as pH, redox reactions, supramolecular complexes, and/or biocatalytically driven reactions. See e.g., Echeverria, et al., Gels (2018), 4, 54; doi:10.3390/gels4020054, which is incorporated herein by reference in its entirety. The type of “releasing agent” or “external trigger” can depend on the type of conditionally removable coating. For example, a conditionally removable coating featuring a redox-responsive hydrogel can be removed upon application of a releasing agent that includes a reducing agent such as dithiothreitol (DTT). As another example, a pH-responsive hydrogel can be removed upon the application of a releasing agent that changes the pH.

In some embodiments, a method disclosed herein comprises de-crosslinking the reversibly cross-linked biological sample. In some embodiments, the de-crosslinking is performed prior to the spatial assay. The de-crosslinking does not need to be complete. In some embodiments, only a portion of crosslinked molecules in the reversibly cross-linked biological sample are de-crosslinked and allowed to migrate.

(ix) Clearing

In some embodiments of the methods provided herein, the method comprises clearing the biological sample embedded within the three-dimensional polymerized matrix. In some instances, the clearing step may comprise the use of a suitable clearing agent, such as an organic solvent, high refractive index aqueous solution, hyperhydrating solution, detergent, digestion enzyme (e.g., proteinase K) or protein denaturant (see, e.g., Ariel (2017), “A Beginner's Guide to Tissue Clearing”, Int. J. Biochem. Cell Biol. 84:35-39, the entire contents of which are incorporated herein by reference).

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

As described herein, the methods of the present disclosure can provided three-dimensional polymerized matrices comprising a biological sample, or biological samples embedded in three-dimensional polymerized matrices, wherein the three-dimensional polymerized matrix has a thickness that is significantly reduced (e.g., less than or equal to about 200 μm) relative to the thickness of similar three-dimensional polymerized matrices, such as hydrogels, prepared using existing methods and apparatus. Due to the extreme thinness of the three-dimensional matrices obtained by the present methods, the time required for clearing of biological samples embedded therein is also reduced. In some embodiments, the clearing is performed for less than about 120 minutes, less than about 60 minutes, less than about 45 minutes, or less than about 30 minutes. In other embodiments, the clearing is performed for less than about 60 minutes or less than about 30 minutes.

### VII. Characterization, Detection and Analysis

In some embodiments, a three-dimensional polymerized matrix disclosed herein and/or a biological sample embedded in the three-dimensional polymerized matrix may be subjected to further characterization, analyte detection and/or analysis.

A. Characterization of Three-Dimensional Polymerized Matrix

As described herein, the methods of the present disclosure provide three-dimensional polymerized matrices or biological samples embedded in three-dimensional polymerized matrices, wherein the three-dimensional polymerized matrices have an average thickness of between about 5 μm and about 200 μm.

In some embodiments, the methods of the present disclosure further comprise measuring the thickness of the three-dimensional polymerized matrix. In some embodiments, the matrix-forming material comprises a plurality of fluorescent beads, and the thickness of the three-dimensional polymerized matrix is measured by imaging the fluorescent beads, e.g., by stepping the focal plane through the matrix and counting the number of well-resolved beads in the z-direction. In some embodiments, the resolution of the thickness measurement may be affected by the size of the fluorescent beads used. In certain embodiments, the fluorescent beads have an average diameter of about 0.2 μm.

B. In Situ Analysis

In some aspects, the provided embodiments of three-dimensional polymerized matrices and biological samples embedded therein can be employed as samples in an in situ method of analyzing nucleic acid sequences, such as in situ hybridization and/or in situ sequencing, for example from intact tissues or samples in which the spatial information has been preserved. Exemplary in situ methods include sequential fluorescent in situ hybridization (e.g., MERFISH, SeqFISH, etc.), ligation-based in situ sequencing (e.g., Sequencing by Dynamic Annealing and Ligation (SEDAL) as described in US 2021/0164039, incorporated herein by reference in its entirety), and hybridization-based in situ sequencing (HybISS) (e.g., as described in US 2021/0340618, incorporated herein by reference in its entirety) or a combination thereof. In some aspects, the embodiments can be applied in an imaging or detection method for multiplexed nucleic acid analysis. In some aspects, the provided embodiments can be used to identify or detect single nucleotides of interest in target nucleic acids. In some aspects, the provided embodiments can be used to crosslink the nucleic acid concatemers via photoreactive nucleotides, e.g., to a complementary strand, to increase the stability of the nucleic acid concatemers probe in situ.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

In some aspects, the provided herein are methods for analyzing, e.g., detecting or determining, the presence of one or more analytes in the biological sample embedded in the three-dimensional polymerized matrix obtained according to the methods described herein. In some aspects, an analyte disclosed herein can include any biological substance, structure, moiety, or component to be analyzed. In some aspects, a target disclosed herein may similarly include any analyte of interest, such as a biomarker.

(i) Analytes

Systems, apparatus, and methods can be used to analyze any number of analytes present in the biological samples of the present disclosure. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample. In some embodiments, each analyte panel comprises 1, 2, 3, 4, 5, 6, 7, 8, 9, 10, or more analytes (e.g., biomarkers). In some embodiments, any one or more of the analyte panels can comprise about 1, about 5, about 10, about 25, about 50, about 100, about 250, about 500, about 1,000, about 2,500, about 5,000 or more analytes (e.g., biomarkers).

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for in situ analysis) to the analytes in the cell or cell compartment or organelle.

Analytes can be broadly classified into one of two groups: nucleic acid analytes, and non-nucleic acid analytes. A method disclosed herein can be used to analyze nucleic acid analytes and/or non-nucleic acid analytes in any suitable combination.

Examples of nucleic acid analytes include DNA analytes such as genomic DNA, methylated DNA, specific methylated DNA sequences, fragmented DNA, mitochondrial DNA, in situ synthesized PCR products, and RNA/DNA hybrids.

Examples of nucleic acid analytes also include RNA analytes such as various types of coding and non-coding RNA. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. Additional examples of RNA analytes include rRNA, tRNA, miRNA, and viral RNA. The RNA can be a transcript (e.g., present in a tissue section). The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In any of the embodiments herein, the method can comprise analyzing one or more non-nucleic acid analytes, such as protein analytes. In some embodiments, each non-nucleic acid analyte is linked to a labelling agent, e.g., an antibody or antigen binding fragment thereof linked to a reporter oligonucleotide.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte.

Cell surface features corresponding to analytes can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

In certain embodiments, an analyte or a complex or product thereof may be immobilized in a sample, e.g., to one or more other molecules within the sample and/or within a matrix, generally at the location of the analyte within a native biological sample, e.g., under a physiological or pathological condition and/or while the sample is live. The analyte or a complex or product thereof may be immobilized within the sample and/or matrix by steric factors. The analyte or a complex or product thereof may also be immobilized within the sample and/or matrix by covalent or noncovalent bonding. In this manner, the analyte or a complex or product thereof may be considered to be attached to the sample or matrix. By being immobilized to the sample and/or matrix, such as by covalent bonding or cross-linking, the size and/or spatial relationship of the analyte or a complex or product thereof can be maintained. By being immobilized to the sample and/or matrix, such as by covalent bonding or cross-linking, the analyte or a complex or product thereof is resistant to movement or unraveling under mechanical stress.

(ii) Labelling Agents

In some embodiments, an analyte labelling agent (also referred to at times as a “capture agent”) may include an agent that interacts with an analyte (e.g., an analyte in a sample) and with a probe to identify the analyte. In some embodiments, the sample may be contacted with one or more labelling agents prior to, during, or after an in situ assay. In some embodiments, the method comprises one or more post-fixing (also referred to as post-fixation) steps after contacting the sample with one or more labelling agents. In some embodiments, the analyte labelling agent comprises an analyte binding moiety and a labelling agent barcode domain (e.g., an analyte binding moiety barcode).

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, an analyte binding moiety may include any molecule or moiety capable of binding to an analyte (e.g., a biological analyte, e.g., a macromolecular constituent). In some embodiments, disclosed herein is a method wherein the analyte binding moiety that binds to a biological analyte can include, but is not limited to, an antibody, or an epitope binding fragment thereof, a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The analyte binding moiety can bind to the macromolecular constituent (e.g., analyte) with high affinity and/or with high specificity. The analyte binding moiety can include a nucleotide sequence (e.g., an oligonucleotide), which can correspond to at least a portion or an entirety of the analyte binding moiety. The analyte binding moiety can include a polypeptide and/or an aptamer (e.g., a polypeptide and/or an aptamer that binds to a specific target molecule, e.g., an analyte). The analyte binding moiety can include an antibody or antibody fragment (e.g., an antigen-binding fragment) that binds to a specific analyte (e.g., a polypeptide).

As used herein, the term “analyte binding moiety barcode” refers to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (i.e., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety, and the cell can be subjected to spatial analysis (e.g., any of the variety of spatial analysis methods described herein). Non-limiting aspects of spatial analysis methodologies are described in U.S. Pat. Nos. 10,308,982; 9,879,313; 9,868,979; Liu et al., bioRxiv 788992, 2020; U.S. Pat. Nos. 10,774,372; 10,774,374; WO 2018/091676; U.S. Pat. Nos. 10,030,261; 9,593,365; 10,002,316; 9,727,810; 10,640,816; Rodrigues et al., Science 363(6434):1463-1467, 2019; Lee et al., Nat. Protoc. 10(3):442-458, 2015; U.S. Pat. Nos. 10,179,932; 10,138,509; Trejo et al., PLoS ONE 14(2):e0212031, 2019; U.S. Patent Application Publication Nos. 2018/0245142; 2019/0177718; Chen et al., Science 348(6233):aaa6090, 2015; Gao et al., BMC Biol. 15:50, 2017; WO 2018/107054; U.S. Patent Application Publication Nos. 2019/0161796; 2020/0224244; 2019/0194709; WO 2011/094669; U.S. Pat. Nos. 7,709,198; 8,604,182; 8,951,726; 9,783,841; 10,041,949; WO 2016/057552; WO 2017/147483; U.S. Pat. Nos. 10,370,698; 10,724,078; 10,364,457; 10,317,321; WO 2018/136856; U.S. Patent Application Publication Nos. 2017/0241911; 2017/0029875; U.S. Pat. No. 10,059,990; WO 2018/057999; and Gupta et al., Nature Biotechnol. 36:1197-1202, 2018, and can be used herein in any combination. Further non-limiting aspects of spatial analysis methodologies are described herein.

(iii) Signal Amplification and/or Detection

In some aspects, a three-dimensional polymerized matrix disclosed herein and/or a biological sample embedded in the three-dimensional polymerized matrix may be subjected to in situ analysis, including in situ sequencing and/or in situ hybridization-based analysis of an intact tissue or non-homogenized tissue.

a. In Situ Signal Amplification

In some aspects, the provided methods involve analyzing, e.g., detecting or determining, one or more sequences (e.g., any of the barcodes described herein) and/or in a product or derivative thereof. In some embodiments, a method disclosed herein may also comprise one or more in situ signal amplification components. In some embodiments, the present disclosure relates to the detection of nucleic acids sequences in situ using probe hybridization and generation of amplified signals associated with the probes, wherein background signal is reduced and sensitivity is increased.

Exemplary signal amplification methods include targeted deposition of detectable reactive molecules around the site of probe hybridization, targeted assembly of branched structures (e.g., as described in US 2022/0064697 incorporated herein by reference, bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER), or any combination thereof. In some embodiments, non-enzymatic signal amplification may be used.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, WO 2019/236841, WO 2020/102094, WO 2020/163397, and WO 2021/067475, all of which are incorporated herein by reference in their entireties.

In some embodiments, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any of the embodiments herein, the target nucleic acid molecule and/or the analyte can be an RCA product.

In some embodiments, detection of nucleic acids sequences in situ includes an assembly for branched signal amplification. In some embodiments, the assembly complex comprises an amplifier hybridized directly or indirectly (via one or more oligonucleotides) to a sequence (e.g., any of the barcodes described herein) present in a probe described herein and/or in a product or derivative thereof. In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some embodiments, the sequences (e.g., any of the barcodes described herein) present in a probe described herein and/or in a product or derivative thereof can be detected in with a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, the sample may be contacted with a plurality of concatemer primers and a plurality of labeled probes. see e.g., U.S. Pat. Pub. No. US20190106733, which is incorporated herein by reference, for exemplary molecules and PER reaction components.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate an amplicon (e.g., a DNA nanoball) containing multiple copies of the circular template or a sequence thereof. Techniques for rolling circle amplification (RCA) include linear RCA, a branched RCA, a dendritic RCA, or any combination thereof (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, U.S. Pat. Nos. 10,138,509, 10,266,888, 10,494,662, and 10,545,075, all of which are incorporated herein by reference. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

b. Signal Detection

In some embodiments, provided herein are methods comprising in situ assays using microscopy as a readout, e.g., nucleic acid sequencing, hybridization, or other detection or determination methods involving an optical readout. In some cases, analysis is performed on one or more images captured, and may comprise processing the image(s) and/or quantifying signals observed. In some embodiments, images of signals from different fluorescent channels and/or detectable probe hybridization (and optionally ligation) cycles can be compared and analyzed. In some embodiments, images of signals (or absence thereof) at a particular location in a sample from different fluorescent channels and/or sequential detectable probe hybridization (and optionally ligation) cycles can be aligned to analyze an analyte at the location. For instance, a particular location in a sample can be tracked and signal spots from sequential hybridization (and optionally ligation) cycles can be analyzed to detect a target polynucleotide sequence (e.g., a barcode sequence or subsequence thereof) in a nucleic acid at the location. The analysis may comprise processing information of one or more cell types, one or more types of analytes, a number or level of analyte, and/or a number or level of cells detected in a particular region of the sample. In some embodiments, the analysis comprises detecting a sequence e.g., a barcode sequence present in an amplification product at a location in the sample. In some embodiments, the analysis includes quantification of puncta (e.g., if amplification products are detected). In some cases, the analysis includes determining whether particular cells and/or signals are present that correlate with one or more analytes from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

In some aspects, detection or determination of a sequence of one, two, three, four, five, or more nucleotides of a target nucleic acid is performed in situ in a cell in an intact tissue. In some embodiments, the assay comprises detecting the presence or absence of an amplification product (e.g., RCA product). In some embodiments, the present disclosure provides methods for high-throughput profiling of a large number of targets in situ, such as transcripts and/or DNA loci, e.g., for detecting and/or quantifying nucleic acids and/or proteins in cells, tissues, organs or organisms.

In some aspects, provided herein is a method comprising analyzing biological targets based on in situ hybridization of probes comprising nucleic acid sequences. In some embodiments, the method comprises sequential hybridization of detectably-labelled oligonucleotides to barcoded probes that directly or indirectly bind to biological targets in a sample. In some embodiments, a detectably-labelled oligonucleotide directly binds to one or more barcoded probes. In some embodiments, a detectably-labelled oligonucleotide indirectly binds to one or more barcoded probes, e.g., via one or more bridging nucleic acid molecules.

In some aspects, an in situ hybridization based assay is used to localize and analyze nucleic acid sequences (e.g., a DNA or RNA molecule comprising one or more specific sequences of interest) within a native biological sample, e.g., a portion or section of tissue or a single cell. In some embodiments, the in situ assay is used to analyze the presence, absence, an amount or level of mRNA transcripts (e.g., a transcriptome or a subset thereof, or mRNA molecules of interest) in a biological sample, while preserving spatial context. In some embodiments, the present disclosure provides compositions and methods for in situ hybridization using directly or indirectly labeled molecules, e.g., complementary DNA or RNA or modified nucleic acids, as probes that bind or hybridize to a target nucleic acids within a biological sample of interest.

Nucleic acid probes, in some examples, may be labelled with radioisotopes, epitopes, hapten, biotin, or fluorophores, to enable detection of the location of specific nucleic acid sequences on chromosomes or in tissues. In some embodiments, probes are locus specific (e.g., gene specific) and bind or couple to specific regions of a chromosome. In alternative embodiments, probes are alphoid or centromeric repeat probes that bind or couple to repetitive sequences within each chromosome. Probes may also be whole chromosome probes (e.g., multiple smaller probes) that bind or couple to sequences along an entire chromosome.

In some embodiments, provided herein is a method comprising DNA in situ hybridization to measure and localize DNA. In some embodiments, provided herein is a method RNA in situ hybridization to measure and localize RNAs (e.g., mRNAs, lncRNAs, and miRNAs) within a biological sample (e.g., a fixed tissue sample). In some embodiments, RNA in situ hybridization involves single-molecule RNA fluorescence in situ hybridization (FISH). In some embodiments, fluorescently labeled nucleic acid probes are hybridized to pre-determined RNA targets, to visualize gene expression in a biological sample. In some embodiments, a FISH method comprises using a single nucleic acid probe specific to each target, e.g., single-molecule FISH (smFISH). The use of smFISH may produce a fluorescence signal that allows for quantitative measurement of RNA transcripts. In some embodiments, smFISH comprises a set of nucleic acid probes, about 50 base pairs in length, wherein each probe is coupled to a set fluorophores. For example, the set of nucleic acid probes may comprise five probes, wherein each probe coupled to five fluorophores. In some embodiments, said nucleic acid probes are instead each coupled to one fluorophore. For example, a smFISH protocol may use a set of about 40 nucleic acid probes, about 20 base pairs in length, each coupled to a single fluorophore. In some embodiments, the length of the nucleic acid probes varies, comprising 10 to 100 base pairs, such as 30 to 60 base pairs. Alternatively, a plurality of nucleic acid probes targeting different regions of the same RNA transcript may be used. It will be appreciated by those skilled in the art that the type of nucleic acid probes, the number of nucleic acid probes, the number of fluorophores coupled to said probes, and the length of said probes, may be varied to fit the specifications of the individual assay.

In some aspects, the provided methods further involve analyzing, e.g., detecting or determining, one or more sequences present in the target nucleic acid and/or in the nucleic acid as described herein. In some embodiments, the detecting comprises hybridizing one or more detectably labeled probes to the nucleic acid concatemer, or via hybridization to adapter probes that hybridize to the nucleic acid concatemer). In some embodiments, the analysis comprises determining the sequence of all or a portion of the nucleic acid concatemer (e.g., a barcode sequence or a complement thereof), wherein the sequence is indicative of a sequence of the target nucleic acid.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization in a FISH-type assay, sequencing by hybridization).

In some embodiments, the methods comprise sequencing all or a portion of the nucleic acid. In some embodiments, the sequence of the nucleic acid, or barcode thereof, is indicative of a sequence of the target nucleic acid to which the nucleic acid is hybridized. In some embodiments, the analysis and/or sequence determination comprises sequencing all or a portion of the nucleic acid and/or in situ hybridization to the nucleic acid. In some embodiments, the sequencing step involves sequencing by hybridization, sequencing by ligation, sequencing by synthesis, sequencing by binding, and/or fluorescent in situ sequencing (FISSEQ), hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US2017/0009278, which is incorporated herein by reference, for exemplary probes and HCR reaction components. In some embodiments, the detection or determination comprises hybridizing to the first overhang a detection oligonucleotide labeled with a fluorophore, an isotope, a mass tag, or a combination thereof. In some embodiments, the detection or determination comprises imaging the probe hybridized to the target nucleic acid (e.g., imaging one or more detectably labeled probes hybridized thereto). In some embodiments, the target nucleic acid is an mRNA in a tissue sample, and the detection or determination is performed when the target nucleic acid and/or the amplification product is in situ in the tissue sample. In some embodiments, the target nucleic acid is an amplification product (e.g., a rolling circle amplification product/nucleic acid).

In some aspects, the provided methods comprise imaging the probe hybridized to the nucleic acid, for example, via binding of one or more detectable probes and detecting signals associated with detectable labels. In some embodiments, the detectable probe comprises a detectable label that can be measured and quantitated. The terms “label” and “detectable label” comprise a directly or indirectly detectable moiety that is associated with (e.g., conjugated to) a molecule to be detected, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more nucleic acid concatemer(s) crosslinked to the complementary strand of the target nucleic acid described herein. In some embodiments, the methods involve incubating the detectable probe containing the detectable label with the sample, washing unbound detectable probe, and detecting the label, e.g., by imaging. In some embodiments, the nucleic acid concatemer(s) remain crosslinked to the target nucleic acid during the washing and detecting steps.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5- UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods for custom synthesis of nucleotides having other fluorophores include those described in Henegariu et al. (2000) Nature Biotechnol. 18:345.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as an Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and PCT publication WO 91/17160. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detectable probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detectable probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECS™), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXS™), and intact tissue expansion microscopy (exM).

In some embodiments, sequencing can be performed in situ and such sequencing methods typically involve incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (i.e., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932. Exemplary techniques for in situ sequencing comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112), and FISSEQ (described for example in US 2019/0032121).

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232.

In some embodiments, sequencing can be performed by sequential fluorescence hybridization (e.g., sequencing by hybridization). Sequential fluorescence hybridization can involve sequential hybridization of detectable probes comprising an oligonucleotide and a detectable label.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597.

In some embodiments, the barcodes of a probe or product thereof can be targeted by one or more other probes, such as unlabeled intermediate probes (which may be targeted by detectably labeled probes) or detectably labeled probes (e.g., fluorescently labeled oligonucleotides). In some embodiments, one or more decoding schemes are used to decode the signals, such as fluorescence, for sequence determination. In any of the embodiments herein, barcodes can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; WO 2018/026873 A1; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### VIII. Kits

Also provided herein are kits for performing the methods of processing a biological sample as described in the present disclosure.

A. Sample Transfer Kit

In one aspect, provided herein is a kit for use in sample transfer of a biological sample to substrate for in situ analysis according to the methods provided herein.

For example, provided herein is a kit for transferring a biological sample from a first substrate to a second substrate, wherein the second substrate comprises a coating with or is functionalized with one or more substances to facilitate attachment of a biological sample and/or a three-dimensional polymerized matrix and/or comprises one or more positioning markers and/or fiducial markers. In one aspect, provided herein is a kit, a first matrix-forming material; a second matrix-forming material; a substrate, wherein: the substrate comprises a coating comprising. or is functionalized with, one or more substances to facilitate attachment of a biological sample and/or a three-dimensional polymerized matrix, and the substrate comprises one or more positioning markers and/or fiducial markers; and optionally, instructions for use thereof.

In some embodiments, the substrate comprises a coating comprising, or is functionalized with, one or more substances to facilitate attachment of a biological sample and/or a three-dimensional polymerized matrix, wherein the one or more substances to facilitate attachment comprise lectins, poly-lysine, antibodies, polysaccharides, or binding moieties capable of covalent binding (e.g., acryloyls), or any combination thereof. In some embodiments, the substrate further comprises a coating comprising, or is functionalized with, one or more substances to deter attachment and wherein the one or more substances to facilitate attachment and the one or more substances to deter attachment are patterned to provide an adhesive region and a non-adhesive region on the surface of the substrate.

In some embodiments, the kit further comprises one or more separation agents. In some embodiments, one or more separation agents comprise a detergent, a salt, a digestion enzyme, or a protein denaturant.

In some embodiments, the first matrix-forming material comprises a matrix-forming material. In some embodiments, the first matrix-forming material comprising a plurality of stimulus-responsive matrix-forming monomers. In some embodiments, the matrix-forming material comprises polyacrylamide, cellulose, alginate, polyamide, cross-linked agarose, cross-linked dextran or cross-linked polyethylene glycol. In some embodiments, the matrix-forming material comprises acrylamide and N,N-methylenebisacrylamide (BIS), In some embodiments, the matrix-forming material comprises acrylamide, polydopamine (PDA) and N,N′-diallyltartardiamide (DATD). In some embodiments, the matrix-forming material comprises one or more cross-linking agents or a plurality of matrix-forming monomers capable of forming cross-linking bonds. In some embodiments, the one or more cross-linking agents comprises N,N,N′,N′-tetramethylethylenediamine (TEMED), N,N-methylenebisacrylamide (BIS), or N,N′-diallyltartardiamide (DATD).

In some embodiments, the kit further comprises one or more cross-linking agents.

B. Sample Adapter Kit

In another aspect, provided herein is a kit for use in adapting a biological sample affixed to substrate for in situ analysis according to the methods and universal adapter provided herein.

For example, provided herein is a kit containing an adapter and a cover for adapting a biological sample immobilized to a non-specialized (e.g., unmodified for in situ analysis) substrate for in situ analysis, wherein the adapter comprises one or more positioning markers and/or fiducial markers. In one aspect, provided herein is a kit comprising: an adapter, comprising a body having a first surface, a second surface, and a first hole extending through the body from the first surface to the second surface, wherein the first surface and/or second surface comprises one or more positioning markers and/or fiducial markers and wherein the second surface is configured to contact and to be supported by a substrate, wherein the first hole is configured in the body to be positioned over the substrate to form a sample well configured to contain a biological sample affixed to the substrate; a cover, comprising: a second body having a third surface, a fourth surface, and a second hole extending through the second body from the third surface to the fourth surface; wherein the first hole and second hole are configured such that when the cover is placed atop the first surface of the adapter, the first hole is positioned centrally under the second hole and the second hole has a cross-sectional area such that the first hole and the one or more positioning markers and/or fiducial markers of the first and/or second surface are visible through the second hole; and optionally, instructions for use thereof.

In some embodiments, the kit is a kit for preparing a sample cassette for in situ analysis.

In some embodiments, the kit comprises a matrix-forming material. In some embodiments, the matrix-forming material comprises polyacrylamide, cellulose, alginate, polyamide, cross-linked agarose, cross-linked dextran or cross-linked polyethylene glycol. In some embodiments, the matrix-forming material comprises acrylamide and N,N-methylenebisacrylamide (BIS), or acrylamide, polydopamine (PDA) and N,N′-diallyltartardiamide (DATD). In some embodiments, the matrix-forming material comprises one or more cross-linking agents or a plurality of matrix-forming monomers capable of forming cross-linking bonds. In some embodiments, the one or more cross-linking agents comprises N,N,N′,N′-tetramethylethylenediamine (TEMED), N,N-methylenebisacrylamide (BIS), or N,N′-diallyltartardiamide (DATD).

C. Additional Kit Components

In some embodiments, the kits can contain reagents and/or consumables required for performing one or more steps of the provided methods. In some embodiments, the kit comprises one or more materials for forming a three-dimensional polymerized matrix, e.g., polymerization initiators and/or spacer materials. In some embodiments, the kit comprises one or more initiators, optionally wherein the one or more initiators comprises potassium peroxodisulfate or ammonium sulfate. In some embodiments, the kit comprises one or more spacers (e.g., adhesive tape, ultrathin silicone gaskets, or microparticles). In other embodiments, the kit comprises one or more components for characterizing the three-dimensional polymerized matrices formed, such as fluorescent beads. In some embodiments, the kit further comprises fluorescent beads, which may be added to a matrix-forming material, optionally wherein the fluorescent beads have an average diameter of about 0.2 μm.

In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as detectable probes or detectable labels. In some embodiments, the kits optionally contain other components, for example nucleic acid primers, enzymes and reagents, buffers, nucleotides, photoreactive nucleotides, and reagents for additional assays.

In still other embodiments, the kits further comprise one or more reagents for performing the analytical methods provided herein. In some embodiments, the kits further comprise one or more reagents required for one or more steps comprising hybridization, ligation, extension, detection, sequencing, and/or sample preparation as described herein. In some embodiments, the kit further comprises a target nucleic acid, e.g., any described in Section III. In some embodiments, any or all of the oligonucleotides are DNA molecules. In some embodiments, the target nucleic acid is a messenger RNA molecule. In some embodiments, the target nucleic acid is a probe (e.g., a padlock probe) or an amplification product thereof (e.g., a rolling circle amplification product, such as a nucleic acid concatemer). The various components of the kit may be present in separate containers or certain compatible components may be pre-combined into a single container. In some embodiments, the kits further contain instructions for using the components of the kit to practice the provided methods.

### IX. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers. Hybridization temperatures can be as low as 5° C., but are typically greater than 22° C., and more typically greater than about 30° C., and typically in excess of 37° C. Hybridizations are often performed under stringent conditions, i.e., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, i.e. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods. “Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The presently disclosed subject matter will be better understood by reference to the following Examples, which are provided as exemplary of the disclosure, and not by way of limitation.

### Example 1: Sample Transfer Method for Transferring Biological Sample from any Slide to Specialized Slide for In Situ Analysis

The present example describes an exemplary method for processing a biological sample by transferring a biological sample affixed to an initial slide to a specialized slide for in situ analysis.

Starting with a tissue section sample already immobilized on an initial slide (slide A), the sample is fixed and permeabilized. A first specialized hydrogel mixture (hydrogel mixture A) is added to the tissue sample and polymerized such that the tissue sample is embedded in the hydrogel matrix formed from hydrogel mixture A. The hydrogel mixture A can contain hydrogel monomers, such as polyacrylamide, cellulose, alginate, polyamide, cross-linked agarose, cross-linked dextran or cross-linked polyethylene glycol. The hydrogel can also optionally contain stimulus-responsive monomers (e.g., acrylic acid and acrylamide monomers, or N-isopropylacrylamide and acrylamide monomers, which can expand upon exposure to the appropriate stimulus and the expansion of which can facilitate detachment from the initial slide, and/or cross-linking agents suitable for forming covalent bonds between the biological sample and the hydrogel matrix (e.g., N,N,N′,N′-tetramethylethylenediamine (TEMED), N,N-methylenebisacrylamide (BIS), or N,N′-diallyltartardiamide (DATD)).

The embedded sample is then cleared with a suitable clearing agent, such as a detergent, digestion enzyme (e.g., proteinase K) or protein denaturant. The embedded sample, if it contains an expanding stimulus-responsive monomer, may be expanded with exposure to the appropriate stimulus (e.g., a salt concentration change and/or temperature change). The embedded sample is transferred to the specialized slide (slide B). The transfer of the embedded sample to the specialized slide is carried out by sandwiching the embedded sample between the initial slide (slide A) and the specialized slide (slide B), inverting the embedded sample so that the specialized slide is underneath the embedded sample, and removing the initial slide. The embedded sample is optionally further agitated (e.g., gently shaken) and/or treated with a separation reagent such as a detergent or salt solution prior to or after the embedded sample is sandwiched between the initial slide and the specialized slide. The separation reagent may be introduced via passive diffusion (e.g., immersion) with or without agitation (e.g., sonication) or other physical disruption, or via active flow (e.g., using a pump to force separation reagent into the sandwiched, embedded biological sample).

Once the embedded sample is supported by the specialized slide and the initial slide has been removed, a second hydrogel mixture (hydrogel mixture B) is added to the embedded tissue sample and polymerized such that the tissue sample is embedded in the hydrogel matrix formed from hydrogel mixture B. The embedded biological sample and hydrogel matrices are optionally further immobilized to the specialized slide during the polymerization process or after polymerization with the addition of fixation reagents.

### Example 2: Adapter Method for Adapting Biological Sample on any Slide for In Situ Analysis

The present example describes an exemplary method for processing a biological sample by using an adapter for in situ analysis with a biological sample affixed to an initial slide.

Starting with a tissue section sample already immobilized on an initial slide, the sample is fixed and permeabilized. An adapter having a through hole configured to the shape, size and location of the biological sample on the initial slide and further having positional markers and/or fiducial markers and/or surface functionalization is obtained. The adapter is placed over the initial slide so that the biological sample sits within the confines of the through hole and any positional markers and/or fiducial markers are oriented facing upward away from the initial slide. A cover having a through hole configured according to the shape, size and location of the through hole in the adapter is obtained. The cover is placed over the adapter so that the positional markers and/or fiducial markers and biological sample are unobstructed and clearly visible through the hole of the cover.

A hydrogel mixture is added to the tissue sample and polymerized such that the tissue sample is embedded in the hydrogel matrix formed from hydrogel mixture. The embedded sample is then optionally processed using other sample processing techniques (e.g., clearing, expanding, etc.).

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. A method for processing a biological sample, comprising:
a) delivering a first matrix-forming material to a biological sample immobilized on a first substrate;
b) forming a first three-dimensional polymerized matrix from the first matrix-forming material, thereby embedding the biological sample in the first three-dimensional polymerized matrix;
c) immobilizing the biological sample embedded in the first three-dimensional polymerized matrix to a second substrate;
d) delivering a second matrix-forming material to the biological sample immobilized on the second substrate; and
e) forming a second three-dimensional polymerized matrix from the second matrix-forming material, thereby embedding the biological sample in the second three-dimensional polymerized matrix.

2. The method of claim 1, further comprising fixing the biological sample prior to the delivering step in a).

3. The method of claim 1 or 2, further comprising permeabilizing the biological sample prior to the delivering step in a).

4. The method of any one of claims 1 to 3, wherein a plurality of molecules in the biological sample are attached to the first three-dimensional polymerized matrix to substantially retain a relative three-dimensional spatial relationship among the molecules.

5. The method of any one of claims 1 to 4, further comprising clearing the biological sample embedded in the first three-dimensional polymerized matrix prior to the immobilizing step in c).

6. The method of claim 5, wherein the clearing step comprises contacting the biological sample with a digestion enzyme.

7. The method of any one of claims 1 to 6, further comprising detaching the biological sample embedded in the first three-dimensional polymerized matrix from the first substrate prior to the immobilizing step in c).

8. The method of claim 7, wherein the detaching step comprises expanding the first three-dimensional polymerized matrix, wherein the expansion facilitates detachment of the biological sample from the first substrate.

9. The method of claim 7 or 8, wherein the detaching step comprises contacting the biological sample embedded in the first three-dimensional polymerized matrix with a separation reagent prior to the immobilizing step in c).

10. The method of claim 9, wherein the separation reagent comprises a detergent, a salt, a digestion enzyme, a protein denaturant, or any combination thereof.

11. The method of any one of claims 7 to 10, wherein the detaching step comprises immersing the biological sample in a separation reagent; applying a shear stress to the biological sample; flowing a separation reagent through the first three-dimensional polymerized matrix; sonicating, shaking, or agitating the biological sample in the presence of the separation reagent, or any combination thereof.

12. The method of any one of claims 7 to 11, wherein the detaching step and clearing the biological sample are performed in a single step.

13. The method of any one of claims 1 to 12, further comprising transferring the detached biological sample to the second substrate prior to the immobilizing step in c).

14. The method of any one of claims 1 to 13, wherein a plurality of molecules in the biological sample are attached to the first and/or second three-dimensional polymerized matrix to substantially retain the relative three-dimensional spatial relationship among the molecules.

15. The method of any one of claims 1 to 14, wherein a surface of the second substrate comprises a coating comprising one or more substances to facilitate attachment of a biological sample and/or a three-dimensional polymerized matrix.

16. The method of any one of claims 1 to 15, wherein a surface of the second substrate is functionalized, optionally with one or more substances, to facilitate attachment of a biological sample or a three-dimensional polymerized matrix.

17. The method of claim 15 or 16, wherein the one or more substances comprise lectins, poly-lysine, antibodies, polysaccharides, or binding moieties capable of covalent binding, or any combination thereof.

18. The method of any one of claims 1 to 17, wherein a surface of the second substrate further comprises a coating comprising one or more substances to deter attachment of a biological sample and/or a three-dimensional polymerized matrix, wherein the one or more substances to facilitate attachment and the one or more substances to deter attachment are patterned to provide an adhesive region and a non-adhesive region on the surface of the second substrate.

19. The method of any one of claims 1 to 18, wherein a surface of the second substrate is functionalized, optionally with one or more substances, to deter attachment of a biological sample and/or a three-dimensional polymerized matrix, wherein the one or more substances to facilitate attachment and the one or more substances to deter attachment are patterned to provide an adhesive region and a non-adhesive region on the surface of the second substrate.

20. The method of any one of claims 1 to 19, wherein a surface of the second substrate has a recessed cavity.

21. The method of claim 20, wherein the recessed cavity comprises a coating or is functionalized with one or more substances to facilitate attachment of a biological sample and/or a three-dimensional polymerized matrix.

22. The method of any one of claims 1 to 21, wherein the biological sample is a tissue slice between about 1 μm and about 50 μm in thickness.

23. The method of any one of claims 1 to 22, wherein the first three-dimensional polymerized matrix and the second three-dimensional polymerized matrix are formed by subjecting the first matrix-forming material and second matrix-forming material to polymerization, respectively.

24. The method of claim 23, wherein the polymerization is initiated by adding a polymerization-inducing catalyst, UV light or functional cross-linkers.

25. The method of any one of claims 1 to 24, wherein the first matrix-forming material and/or second matrix-forming material comprises polyacrylamide, cellulose, alginate, polyamide, cross-linked agarose, cross-linked dextran or cross-linked polyethylene glycol.

26. The method of any one of claims 1 to 25, wherein the first matrix-forming material comprises a plurality of stimulus-responsive matrix-forming monomers and wherein the first three-dimensional polymerized matrix expands when exposed to a suitable stimulus.

27. The method of claim 26, wherein the plurality of stimulus-responsive matrix-forming monomers comprises acrylic acid and acrylamide monomers, or N-isopropylacrylamide and acrylamide monomers.

28. The method of claim 26 or claim 27, further comprising expanding the biological sample embedded in the first three-dimensional polymerized matrix prior to the immobilizing step.

29. The method of claim 28, wherein the expanding step comprises subjecting the first three-dimensional polymerized matrix to a change in salt concentration or a temperature change.

30. The method of any one of claims 1 to 29, wherein the first matrix-forming material comprises acrylamide and N,N-methylenebisacrylamide (BIS), or acrylamide, polydopamine (PDA) and N,N′-diallyltartardiamide (DATD).

31. The method of any one of claims 1 to 30, wherein the first matrix-forming material comprises a plurality of matrix-forming monomers comprising functional groups capable of forming covalent bonds with one or more molecules in the biological sample.

32. The method of claim 31, further comprising forming covalent bonds between the biological sample and the first three-dimensional polymerized matrix in which the biological sample is embedded.

33. The method of any one of claims 1 to 32, wherein the second matrix-forming material comprises one or more cross-linking agents or a plurality of matrix-forming monomers capable of forming cross-linking bonds.

34. The method of claim 33, wherein the one or more cross-linking agents comprises N,N,N′,N′-tetramethylethylenediamine (TEMED), N,N-methylenebisacrylamide (BIS), or N,N′-diallyltartardiamide (DATD).

35. The method of claim 33 or 34, wherein the second matrix-forming material comprises one or more initiators.

36. The method of any one of claims 33-35, wherein the plurality of matrix-forming monomers capable of forming cross-linking bonds comprises matrix-forming monomers comprising Click-compatible moieties.

37. The method of any one of claims 1 to 36, wherein one or more substances that facilitate sample attachment are present on the second substrate and/or in the second three-dimensional polymerized matrix.

38. The method of any one of claims 1 to 37, further comprising:
sandwiching the biological sample embedded in the first three-dimensional polymerized matrix between the first substrate and the second substrate; and
removing the first substrate.

39. The method of any one of claims 1 to 38, wherein the first delivering step a) comprises delivering the first matrix-forming material to a first space between a surface of the first substrate and a surface of the second substrate, wherein:
the first space is at least partially enclosed by a first spacer between the first and second substrates,
the first space contains the biological sample immobilized on the surface of the first substrate, and
the first three-dimensional polymerized matrix formed in step b) is sandwiched between the first substrate and the second substrate.

40. The method of claim 39, wherein the first spacer is non-integral to the first or second substrate.

41. The method of any one of claims 1 to 40, wherein the second delivering step d) comprises delivering the second matrix-forming material to a second space between a surface of the second substrate and a surface of a third substrate, wherein
the second space is at least partially enclosed by a second spacer between the second and third substrates, and
the second space contains the biological sample embedded in the first three-dimensional polymerized matrix.

42. The method of claim 41, wherein the second spacer is non-integral to the second or third substrate.

43. The method of any of claims 39-42, wherein the first spacer and/or second spacer is an adhesive tape having a thickness between about 10 μm and about 20 μm.

44. The method of any of claims 39-42, wherein the first spacer and/or second spacer comprises a plurality of microparticles having an average diameter between about 10 μm and 50 μm.

45. The method of any of claims 1-44, further comprising analyzing one or more analytes in the biological sample embedded in the first three-dimensional polymerized matrix and/or the second three-dimensional polymerized matrix in situ on the second substrate.

46. A sample for in situ analysis obtained according to the method of any one of claims 1-45.

47. A sample for in situ analysis, comprising:
a biological sample;
a first three-dimensional polymerized matrix;
a second three-dimensional polymerized matrix; and
a substrate,
wherein the biological sample is embedded in the first three-dimensional polymerized matrix, wherein the first three-dimensional polymerized matrix is further embedded in the second three-dimensional polymerized matrix, and wherein the second three-dimensional polymerized matrix is immobilized to the substrate.

48. The sample of claim 47, wherein the substrate comprises one or more positioning markers and/or fiducial markers on a same surface to which the second three-dimensional polymerized matrix is immobilized.

49. A kit, comprising:
a first matrix-forming material;
a second matrix-forming material;
a substrate, wherein:
the substrate comprises a coating with or is functionalized with one or more substances to facilitate attachment of a biological sample and/or a three-dimensional polymerized matrix, and
the substrate comprises one or more positioning markers and/or fiducial markers; and
optionally, instructions for use thereof.

50. The kit of claim 49, wherein the one or more substances to facilitate attachment comprise lectins, poly-lysine, antibodies, polysaccharides, or binding moieties capable of covalent binding, or any combination thereof.

51. The kit of claim 49 or 50, wherein the substrate further comprises a coating with or is functionalized with one or more substances to deter attachment, and wherein the one or more substances to facilitate attachment and the one or more substances to deter attachment are patterned to provide an adhesive region and a non-adhesive region on the surface of the substrate.

52. The kit of any one of claims 49 to 51, further comprising one or more separation agents.

53. The kit of claim 52, wherein the one or more separation agents comprise a detergent, a salt, a digestion enzyme, or a protein denaturant.

54. The kit of any one of claims 49 to 53, wherein the first matrix-forming material comprises a plurality of stimulus-responsive matrix-forming monomers.

55. The kit of any one of claims 49 to 54, further comprising one or more cross-linking agents.

56. An adapter for a biological sample affixed to a substrate for in situ analysis, comprising:
a body having a first surface, a second surface, and at least one hole extending through the body from the first surface to the second surface;
wherein the first surface comprises one or more positioning markers and/or fiducial markers and wherein the second surface is configured to contact and to be supported by a substrate,
wherein the at least one hole is configured in the body to be positioned over the substrate to form at least one sample well, wherein at least one biological sample or a portion thereof is affixed to the substrate and is contained within a sample well.

57. The adapter of claim 56, wherein the adapter comprises a planar body and the second surface is configured to contact and be supported by a planar substrate.

58. The adapter of claim 56 or 57, wherein the body of the adapter has a thickness of between about 1 μm and 200 μm.

59. The adapter of any of claims 56 to 58, wherein the body of the adapter has a thickness greater than or equal to the thickness of the at least one biological sample, optionally wherein the at least one biological sample has a thickness between 1 μm and 50 μm or between 5 μm and 20 μm.

60. The adapter of any one of claims 56 to 59, wherein the outer perimeter of the body of the adapter is substantially the same as the outer perimeter of the substrate.

61. The adapter of any one of claims 56 to 60, wherein the first surface of the adapter comprises a coating with or is functionalized with one or more substances to facilitate attachment of a biological sample (e.g., tissue sample) and/or a three-dimensional polymerized matrix.

62. The adapter of any one of claims 56 to 61, wherein the at least one hole comprises inner walls, and wherein the inner walls of the at least one hole comprise a coating with, or are functionalized with, one or more substances to facilitate attachment of a biological sample and/or a three-dimensional polymerized matrix.

63. The adapter of claim 61 or 62, wherein the one or more substances to facilitate attachment comprise lectins, poly-lysine, antibodies, polysaccharides, or binding moieties capable of covalent binding, or any combination thereof.

64. The adapter of any one of claims 56 to 63, wherein the first surface and/or the inner walls of the at least one hole of the adapter comprises a coating with, or is functionalized with, one or more substances to deter attachment of one or more analytes, a biological sample (e.g., tissue sample) and/or a three-dimensional polymerized matrix.

65. The adapter of any one of claims 56 to 64, wherein the second surface comprises a coating or is functionalized with one or more substances to facilitate attachment to a biological sample (e.g., tissue sample) and/or a three-dimensional polymerized matrix.

66. The adapter of any one of claims 56 to 65, wherein the adapter is made of glass, an elastomer, or adhesive tape, optionally wherein the elastomer is polydimethylsiloxane (PDMS).

67. A method of processing a biological sample, comprising:
a) applying an adapter to a substrate,
wherein a biological sample is affixed to the substrate,
wherein the adapter comprises a body having a first surface, a second surface, and a first hole extending through the body from the first surface to the second surface;
wherein the first surface comprises one or more positioning markers and/or fiducial markers,
wherein the second surface is in contact with the substrate,
wherein the first hole is configured to be positioned over the substrate to form a sample well, wherein the biological sample is contained within the sample well;
b) delivering a matrix-forming material to the sample well containing the biological sample; and
c) forming a three-dimensional polymerized matrix from the matrix-forming material, thereby embedding the biological sample in the three-dimensional polymerized matrix.

68. The method of claim 67, wherein the substrate to which the biological sample is affixed does not have positioning markers and/or fiducial markers.

69. The method of claim 67 or 68, wherein the adapter comprises a planar body and the second surface is configured to contact and be supported by a planar substrate.

70. The method of any one of claims 67 to 69, wherein the body of the adapter has a thickness of between about 1 μm and 200 μm.

71. The method of any one of claims 67 to 70, wherein the body of the adapter has a thickness greater than or equal to the thickness of the biological sample, optionally wherein the biological sample has a thickness between 1 μm and 50 μm or between 5 μm and 20 μm.

72. The method of any one of claims 67 to 71, wherein the outer perimeter of the body of the adapter is substantially the same as the outer perimeter of the substrate.

73. The method of any one of claims 67 to 72, further comprising applying a cover to the first surface of the adapter, prior to step b) or after step c),
wherein the cover comprises a second body,
wherein the second body comprises a third surface, a fourth surface, and a second hole extending through the second body from the third surface to the fourth surface, and
wherein the biological sample and the first hole are positioned centrally under the second hole and the second hole has a cross-sectional area such that the biological sample, the first hole, the one or more positioning markers and/or fiducial markers of the first surface are visible through the second hole.

74. The method of claim 73, wherein the outer perimeter of the cover is substantially the same as the body of the adapter.

75. The method of any one of claims 67 to 74, wherein the first surface of the adapter comprises a coating with, or is functionalized with, one or more substances to facilitate attachment of a biological sample (e.g., tissue sample) and/or a three-dimensional polymerized matrix.

76. The method of any one of claims 67 to 75, wherein the first hole comprises inner walls, and wherein the inner walls of the first hole comprise a coating with, or are functionalized with, one or more substances to facilitate attachment of a biological sample (e.g., tissue sample) and/or a three-dimensional polymerized matrix.

77. The method of any one of claims 73 to 76, wherein the second hole comprises inner walls, and wherein the inner walls of the second hole comprise a coating with, or are functionalized with, one or more substances to facilitate attachment of a three-dimensional polymerized matrix.

78. The method of any one of claims 75 to 77, wherein the one or more substances to facilitate attachment comprise lectins, poly-lysine, antibodies, polysaccharides, or binding moieties capable of covalent binding, or any combination thereof.

79. The method of any one of claims 67 to 78, wherein the first surface and/or the inner walls of the at least one hole of the adapter comprises a coating with, or is functionalized with, one or more substances to deter attachment of one or more analytes, a biological sample (e.g., tissue sample) and/or a three-dimensional polymerized matrix.

80. The method of any one of claims 67 to 79, wherein the adapter is made of glass, an elastomer, or adhesive tape, optionally wherein the elastomer is polydimethylsiloxane (PDMS).

81. The method of any one of claims 67 to 80, the matrix-forming material comprises a plurality of fluorescent beads, and wherein the thickness of the three-dimensional polymerized matrix is measured by imaging the fluorescent beads.

82. The method of any one of claims 67 to 81, wherein the three-dimensional polymerized matrix is formed by subjecting the matrix-forming material to polymerization.

83. The method of claim 82, wherein the polymerization is initiated by adding a polymerization-inducing catalyst, UV light or functional cross-linkers.

84. The method of any one of claims 67 to 83, wherein the matrix-forming material comprises polyacrylamide, cellulose, alginate, polyamide, cross-linked agarose, cross-linked dextran or cross-linked polyethylene glycol.

85. The method of any of claims 67 to 84, further comprising crosslinking the biological sample embedded in the three-dimensional polymerized matrix.

86. The method of any of claims 67 to 85, further comprising clearing the biological sample embedded within the three-dimensional polymerized matrix.

87. A biological sample for in situ analysis obtained according to the method of any one of claims 67 to 86.

88. A sample cassette for in situ analysis, comprising:
a biological sample affixed to a substrate;
an adapter, comprising a body having a first surface, a second surface, and a first hole extending through the body from the first surface to the second surface,
wherein the first surface comprises one or more positioning markers and/or fiducial markers and wherein the second surface is in contact with and supported by the substrate;
wherein the first hole is configured in the body to be positioned over the substrate to form a sample well configured to contain the biological sample affixed to the substrate; and
a cover, comprising:
a second body having a third surface, a fourth surface, and a second hole extending through the second body from the third surface to the fourth surface,
wherein one of the third surface and the fourth surface is in contact with and supported by the second surface of the adapter, and
wherein the first hole and second hole are configured such that when the cover is placed atop the first surface of the adapter, the first hole is positioned centrally under the second hole and the second hole has a cross-sectional area such that the first hole and the one or more positioning markers and/or fiducial markers of the first surface are visible through the second hole.

89. A kit, comprising:
an adapter, comprising:
a body having a first surface, a second surface, and a first hole extending through the body from the first surface to the second surface,
wherein the first surface comprises one or more positioning markers and/or fiducial markers and wherein the second surface is configured to contact and to be supported by a substrate,
wherein the first hole is configured in the body to be positioned over the substrate to form a sample well configured to contain a biological sample affixed to the substrate;
a cover, comprising:
a second body having a third surface, a fourth surface, and a second hole extending through the second body from the third surface to the fourth surface;
wherein the first hole and second hole are configured such that when the cover is placed atop the first surface of the adapter, the first hole is positioned centrally under the second hole and the second hole has a cross-sectional area such that the first hole and the one or more positioning markers and/or fiducial markers of the first surface are visible through the second hole; and
optionally, instructions for use thereof.


SEQUENCE LISTING (3 sequences)

SEQ ID NO: 1 - protein, 16 aa, synthetic construct
RADARADARADARADA

SEQ ID NO: 2 - protein, 16 aa, synthetic construct
AEAEAKAKAEAEAKAK

SEQ ID NO: 3 - protein, 12 aa, synthetic construct
KLDLKLDLKLDL